(19) United States
(12) Patent Application Publication
(10) Pub. No.: US 2012/0303043 A1
(43) Pub. Date: Nov. 29, 2012

(54) Medical Device Inserters and Processes of Inserting and Using Medical Devices

(75) Inventor: Manuel Luis Donnay, San Francisco, CA (US)

(73) Assignee: Abbott Diabetes Care Inc., Alameda, CA (US)

(21) Appl. No.: 13/407,750

(22) Filed: Feb. 28, 2012

Related U.S. Application Data
(60) Provisional application No. 61/447,607, filed on Feb. 28, 2011.

(51) Int. Cl. A61N 1/05; A61B 17/34

(52) U.S. Cl. 606/129

(57) ABSTRACT

An apparatus for insertion of a medical device in the skin of a subject is provided, as well as methods of inserting medical devices.

### RELATED APPLICATION

The present application claims priority to U.S. provisional application No. 61/447,607 filed Feb. 28, 2011, entitled “Medical Device Inserters and Processes of Inserting and Using Medical Devices”, the disclosure of which is incorporated herein by reference for all purposes.

### BACKGROUND

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type 1 Diabetes) or does not properly utilize insulin (a condition known as Type 2 Diabetes), the blood glucose remains in the blood, resulting in hyperglycemia or abnormally high blood sugar levels.

The fluctuations in blood glucose levels in people suffering from diabetes cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

One way to manage blood glucose levels is testing and monitoring blood glucose levels by using conventional in vitro techniques, such as drawing blood samples, applying the blood to a test strip, and determining the blood glucose level using colorimetric, electrochemical, or photometric test meters. Another more recent technique for monitoring blood glucose levels is by using an in vivo glucose-monitoring system, that continuously and/or automatically tests glucose, such as, for example, the FreeStyle Navigator® Continuous Glucose Monitoring System, manufactured by Abbott Diabetes Care Inc. Unlike conventional blood glucose meters, continuous analyte-monitoring systems employ a sensor at least a portion of which is positioned in a subject, which detects and monitors blood glucose levels. Prior to each use of a new sensor, the user self-inserts at least a portion of the sensor under his/her skin. An inserter assembly may be employed to insert the sensor in the body of the user. In this manner, an introducer sharp, while engaged to the sensor, pierces an opening into the skin of the user, releases the sensor, and is removed from the body of the user. Accordingly, there exists a need for an easy-to-use, simple, insertion assembly that is reliable, minimizes pain, and is easy to use.

### SUMMARY

Sensors for detecting a physical parameter of a subject are disclosed herein. In some embodiments, a sensor is used to determine a level of analyte, such as, e.g., glucose. Devices are disclosed for inserting a sensor into a subject, e.g., at least partially beneath the skin of a subject. Sensor assemblies that include a medical device, such as a sensor and/or an infusion device, and a device to position at least a portion of the medical device are provided, as well as methods of positioning at least a portion of a medical device such as a sensor (e.g., a glucose sensor) and/or an infusion device, and methods of using a positioned medical device, e.g., methods for analyte testing are disclosed herein.

These and other features, objects, and advantages of the disclosed subject matter will become apparent to those persons skilled in the art upon reading the detailed description as more fully described below.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic view of the system in accordance with one embodiment of the disclosed subject matter;

FIG. 2 illustrates analyte monitoring system for real time analyte (e.g., glucose) measurement, data acquisition and/or processing in certain embodiments;

FIG. 3 is a view of an analyte sensor in accordance with certain embodiments of the present disclosure;

FIG. 4 is a sectional, perspective view of an embodiment of an inserter in accordance with the disclosed subject matter;

FIGS. 5-6 are perspective views of components of the inserter of FIG. 4 in accordance with the disclosed subject matter;

FIG. 7 is a sectional, perspective view of a component of the inserter of FIG. 4 in accordance with the disclosed subject matter;

FIGS. 8-9 are perspective views of components of the inserter of FIG. 4 in accordance with the disclosed subject matter;

FIG. 10 is a sectional view of the component of FIG. 4 in accordance with the disclosed subject matter;

FIGS. 11-12 are schematic views of a needle hub in accordance with one embodiment of the disclosed subject matter;

FIG. 13 is a distal end view of a sharp in accordance with one embodiment of the disclosed subject matter;

FIG. 14 is a side view of a sharp in accordance with one embodiment of the disclosed subject matter;

FIG. 15 is a side view of a sharp in accordance with one embodiment of the disclosed subject matter;

FIG. 16 is a perspective view of an inserter in accordance with one embodiment of the disclosed subject matter;

FIG. 17 is a perspective view with parts separated of an inserter in accordance with one embodiment of the disclosed subject matter;

FIG. 18 is an enlarged sectional view with parts separated of an inserter in accordance with one embodiment of the disclosed subject matter;

FIGS. 19-21 are perspective views of components of the inserter of FIG. 4 in accordance with the disclosed subject matter;

FIGS. 22-23 are sectional views of the inserter of FIG. 4 in accordance with the disclosed subject matter;

FIGS. 24-25 are sectional views of components of the inserter of FIG. 4 in accordance with the disclosed subject matter;

FIG. 26 is a sectional view of components of the inserter of FIG. 4 shown separated from the on-body unit in accordance with the disclosed subject matter;

FIG. 27 is a perspective view of another inserter in accordance with the disclosed subject matter;

FIGS. 28-35 are additional views of the components of the inserter of FIG. 27 in accordance with the disclosed subject matter;

FIGS. 36-41 are cross-sectional views of the inserter of FIG. 27 in accordance with the disclosed subject matter;

FIG. 42 is a perspective view with parts separated of an embodiment of a component of the analyte monitoring system in accordance with the disclosed subject matter;

FIG. 43 is a sectional view of the component of FIG. 42 in accordance with the disclosed subject matter;

FIG. 44 is a perspective view with parts separated of another embodiment of a component of the analyte monitoring system in accordance with the disclosed subject matter;

FIG. 45 is a perspective view with parts separated of another embodiment of a component of the analyte monitoring system in accordance with the disclosed subject matter;

FIG. 46A is a perspective view with parts separated of another embodiment of a component of the analyte monitoring system in accordance with the disclosed subject matter;

FIG. 46B is a perspective view with parts separated of another embodiment of a component of the analyte monitoring system in accordance with the disclosed subject matter;

FIGS. 47A-47F are views of another inserter in accordance with the disclosed subject matter;

FIGS. 48A-48F are views of another inserter in accordance with the disclosed subject matter;

FIGS. 49A-49F are views of another inserter in accordance with the disclosed subject matter;

FIGS. 50A-50F are views of another inserter in accordance with the disclosed subject matter;

FIGS. 51A-51G are views of another inserter in accordance with the disclosed subject matter;

FIGS. 52A-52F are views of another inserter in accordance with the disclosed subject matter;

FIGS. 53A-53F are views of another inserter in accordance with the disclosed subject matter; and

FIGS. 54A-54F are views of another inserter in accordance with the disclosed subject matter.

### DETAILED DESCRIPTION OF THE EMBODIMENTS

A detailed description of the disclosure is provided herein. It should be understood, in connection with the following description, that the subject matter is not limited to particular embodiments described, as the particular embodiments of the subject matter may, of course, vary. It is also to be understood that the terminology used herein is for the purpose of describing particular embodiments only, and is not intended to be limiting, since the scope of the disclosed subject matter will be limited only by the appended claims.

Where a range of values is provided, it is understood that each intervening value between the upper and lower limit of that range, and any other stated or intervening value in that stated range, is encompassed within the disclosed subject matter. Every range stated is also intended to specifically disclose each and every “subrange” of the stated range. That is, each and every range smaller than the outside range specified by the outside upper and outside lower limits given for a range, whose upper and lower limits are within the range from said outside lower limit to said outside upper limit (unless the context clearly dictates otherwise), is also to be understood as encompassed within the disclosed subject matter, subject to any specifically excluded range or limit within the stated range. Where a range is stated by specifying one or both of an upper and lower limit, ranges excluding either or both of those stated limits, or including one or both of them, are also encompassed within the disclosed subject matter, regardless of whether or not words such as “from,” “to,” “through,” or “including” are or are not used in describing the range.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

All publications mentioned in this disclosure are, unless otherwise specified, incorporated by reference herein for all purposes, including without limitation to disclose and describe the methods and/or materials in connection with which the publications are cited.

The publications discussed herein are provided solely for their disclosure prior to the filing date of the present application. Nothing herein is to be construed as an admission that the present disclosed subject matter is not entitled to antedate such publication by virtue of prior invention. Further, the dates of publication provided may be different from the actual publication dates, which may need to be independently confirmed.

As used herein and in the appended claims, the singular forms “a,” “an,” and “the” include plural referents unless the context clearly dictates otherwise.

Nothing contained in the Abstract or the Summary should be understood as limiting the scope of the disclosure. The Abstract and the Summary are provided for bibliographic and convenience purposes and due to their formats and purposes should not be considered comprehensive.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosed subject matter. Any recited method can be carried out in the order of events recited, or in any other order which is logically possible. Reference to a singular item includes the possibility that there are plural of the same item present. When two or more items (for example, elements or processes) are referenced by an alternative “or,” this indicates that either could be present separately or any combination of them could be present together except where the presence of one necessarily excludes the other or others.

### System Overview

Embodiments include analyte monitors that are provided in small, lightweight, battery-powered and electronically-controlled systems. Such systems may be configured to detect physical parameters of subjects, such as signals indicative of in vivo analyte levels using an electrochemical sensor, and collect such signals, with or without processing. The electrochemical sensors may employ any suitable measurement technique, e.g., may detect current, may employ potentiometry, etc. Techniques may include, but are not limited to amperometry, coulometry, and voltammetry. In some embodiments, sensing systems may be optical, colorimetric, and the like. In some embodiments, the portion of the system that performs this initial processing may be configured to provide the raw or at least initially processed data to another unit for further collection and/or processing. Such provision of data may be effected, for example, by a wired connection, such as an electrical, or by a wireless connection, such as an infrared (IR) or radio frequency (RF) connection.

Certain embodiments of analyte-monitoring systems for in vivo measurement employ a sensor that measures analyte levels in interstitial fluids at least partially under the surface of the subject's skin. A sensor in such a system may operate as an electrochemical cell. Such a sensor may use any of a variety of electrode configurations, such as a three-electrode configuration (e.g., with “working,” “reference,” and “counter” electrodes) driven by a controlled potential (potentiostat) analog circuit; a two-electrode system configuration (e.g., with only working and counter electrodes or a working electrode and a counter/reference electrode), which may be self-biasing and/or self-powered; and/or other configurations (see, e.g., U.S. patent application Ser. No. 12/393,921, the disclosure of which is incorporated by reference herein for all purposes). In some embodiments, the sensor may be positioned within a blood vessel.

In certain systems, the analyte sensor is in communication with sensor electronics. As used in this disclosure, an on-body unit sometimes refers to such a combination of an analyte sensor with such sensor electronics. The on-body unit may include a housing in which the sensor electronics and at least a portion of the sensor are received.

Certain embodiments are modular. The on-body unit may be separately provided as a physically distinct assembly from a monitor unit, e.g., which displays or otherwise indicates analyte levels to a user. The on-body unit may be configured to provide the analyte levels detected by the sensor and/or other information (such as temperature, sensor life, etc.) over a communication link to the monitor unit. The monitor unit, in some embodiments, may include, e.g., a mobile telephone device, an in vitro glucose meter, a personal digital assistant, or other consumer electronics such as MP3 device, camera, radio, personal computer, etc., or other communication-enabled data-processing device.

The monitor unit may perform a variety of functions such as but not limited to data storage and/or processing and/or analysis and/or communication, etc., on the received analyte data to generate information pertaining to the monitored analyte levels and/or process the other information. The monitor unit may incorporate a display screen, which can be used, for example, to display measured analyte levels, and/or an audio component such as a speaker to audibly provide information to a user, and/or a vibration device to provide tactile feedback to a user. It is also useful for a user of an analyte-monitoring system to be able to see trend indications (including the magnitude and direction of any ongoing trend, e.g., the rate of change of an analyte or other parameter, and the amount of time a subject is above and/or below a threshold, such as a hypoglycemic and/or hyperglycemic threshold, etc.); such data may be displayed either numerically, or by a visual indicator such as an arrow that may vary in visual attributes, like size, shape, color, animation, or direction. The monitor unit may further be adapted to receive information from or about an in vitro analyte test strip, which may be manually or automatically entered into the monitor unit. In some embodiments a monitor unit may incorporate an in vitro analyte test strip port and related electronics in order to be able to make discrete (e.g., blood glucose) measurements using an in vitro test strip (see, e.g., U.S. Pat. No. 6,175,752, the disclosure of which is incorporated by reference herein for all purposes).

The modularity of these systems may vary where one or more components may be constructed to be single use and one or more may be constructed to be re-useable. In some embodiments the sensor is designed to be attachable and detachable from the sensor electronics (and the on-body unit may be reusable), e.g., so that one or more of the components may be reused one or more times, while in other embodiments, the sensor and sensor electronics may be provided as an integrated, undetachable package, which may be designed to be disposable after use, i.e., not re-used.

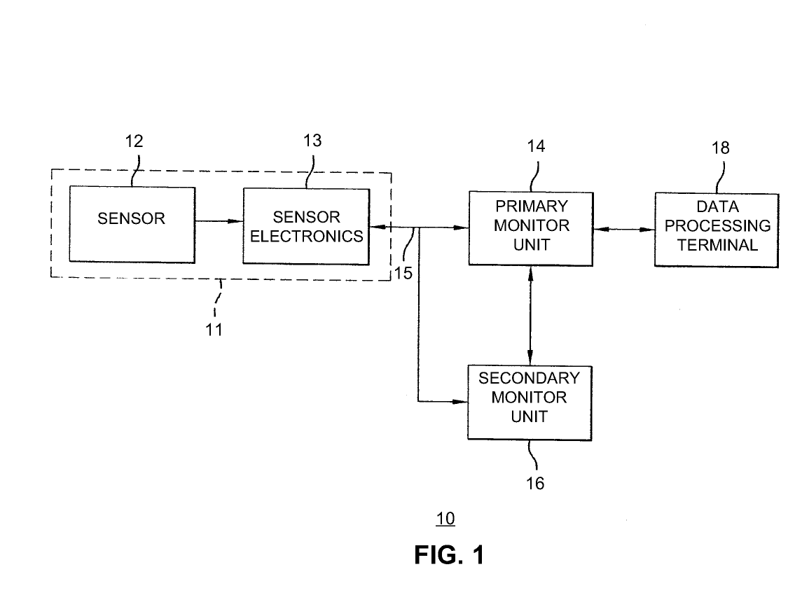

FIG. 1 shows one embodiment of an analyte measurement system 10. In such a system, a data-processing unit or sensor electronics 13 may interact with an analyte sensor 12 to obtain signals representative of analyte levels. Sensor electronics 13 may further include communications circuitry with associated electronics (not shown). In some embodiments, the sensor electronics 13 and at least a portion of sensor 12 are configured to be maintained “on the body” of the subject, in the substantially same position on the body of the subject, for a period of time that may include hours, days, weeks, or a month or more. Accordingly, the sensor electronics 13 and sensor 12 may be referred to collectively herein as an on-body unit 11. A monitor unit 14 may also be provided. In the embodiment shown, sensor electronics 13 and monitor unit 14 communicate via connection 15 (in this embodiment, a wireless radio frequency (RF) connection). Communication may occur, e.g., via RF communication, infrared communication, Bluetooth® communication, Zigbee communication, 802.1x communication, or WiFi communication, etc. In some embodiments, the communication may include a radio frequency of 433 MHz, 13.56 MHz, or the like. In some embodiments, communication between sensor electronics 13 and monitor unit 14 may include radio frequency identification (RFID) techniques, and may be active RFID or passive RFID, where in some embodiments passive RFID technology and the respective system components include the necessary components therefor. For example, in certain embodiments, the on-body unit 11 and the monitor unit 14 may be configured to communicate using RFID communication protocol, wherein the on-body unit 11 may be configured to communicate or provide analyte-related signals to the monitor unit 14 based on the presence or reception of at least one signal from the monitor unit 14. That is, in certain embodiments, the monitor unit 14 may be configured to provide or radiate RF power signals, and when the on-body unit 11 is positioned within the radiated or provided RF signal range radiated from the monitor unit 14, the on-body unit 11 in certain embodiments is configured to provide one or more signals associated with the monitored analyte level received from the analyte sensor 12 to the monitor unit 14. For example, in one embodiment, the monitor unit 14 may include a backscatter RFID reader configured to transmit an RF field such that when the on-body unit 11 is within the transmitted RF field, an antenna is tuned and in turn provides a reflected or response signal (for example, a backscatter signal) to the monitor unit 14. The reflected or response signal may include sampled analyte level data from the analyte sensor. Additional exemplary details for various embodiments can be found in, e.g., U.S. patent application Ser. No. 12/698,124 filed Feb. 1, 2010, the disclosure of which is incorporated by reference herein for all purposes.

In some embodiments, a secondary monitor unit 16 may be provided. A data-processing terminal 18 may also be included, e.g., for providing further processing or review of analyte data. In some embodiments, the secondary monitor unit 16 may include similar components and/or functionalities as the monitor unit 14, or alternatively, include more or less functionalities or components compared to the monitor unit 14 to, for example, analyze, process, store, and/or communicate data from the analyte sensor 12 and/or monitor unit 14.

In some embodiments, system 10 may be a continuous analyte monitor (e.g., a continuous glucose-monitoring system or CGM), and accordingly operate in a mode in which the communications via connection 15 has sufficient range to support a flow of data from on-body unit 11 to monitor unit 14. In some embodiments, the data flow in a CGM system is automatically provided by the on-body unit 11 to the monitor unit 14. For example, no user intervention may be required for the on-body unit 11 to send the data to the monitor unit 14, e.g., it may be continually and/or automatically broadcasted. In some embodiments, the on-body unit 11 provides signal relating to analyte level to the monitor unit 14 automatically and periodically. For example, one or more signals may be provided, e.g., automatically sent, on a schedule, e.g., once every 250 ms, once a second, once a minute, etc. That is, in certain embodiments, the on-body unit 11 may be configured and/or programmed to communicate or transmit the data acquired from the analyte sensor 12 based on a predetermined schedule, and continue to broadcast or transmit signals related to the real time monitored analyte levels based on the predetermined schedule. In such embodiments, the monitor unit 14 may be configured to receive and/or anticipate and acquire the transmitted signals from the on-body unit 11 based on the predetermined schedule, for example, for further processing, such as display, storing, analysis, and/or further data communication.

In some embodiments, one or more signals may be provided to the monitor unit 14 upon the occurrence of, or impending occurrence of, an event, e.g., a hyperglycemic event or a hypoglycemic event, etc. In some embodiments, the one or more signals may be provided to the monitor unit 14 on an adaptive schedule. For example, the schedule for automatically communicating the one or more signals may be adjusted by machine learning techniques (e.g., supervised learning techniques, unsupervised learning techniques, data mining, etc.) according to parameters received by the sensor electronics 13 and/or monitor unit 14, such as, e.g., analyte values and trends, activity levels, meal times, etc.). In some embodiments, sensor electronics 13 may further include local memory in which it may record “logged data” or buffered data collected over a period of time and provide some or all of the accumulated data to monitor unit 14 from time-to-time. Or, a separate data-logging unit may be provided to acquire periodically provided data from sensor electronics 13. Data transmission in a CGM system may be one-way communication, e.g., the on-body unit 11 provides data to the monitor unit 14 without receiving signals from the monitor unit 14. In some embodiments, two-way communication is provided between the on-body unit 11 and the monitor unit 14, using, for example, one or more transceivers or other bi-directional communication components. Such two-way communication may be achieved in certain embodiments independently. That is, in certain embodiments, certain data communication from the on-body unit 11 may be independent of the data received from the monitor unit 14. In other embodiments, certain data communication from the on-body unit 11 may in response or responsive to the data, signal, request, or one or more commands received from the monitor unit 14.

In some embodiments, one or more signals may be provided to the monitor unit 14 from the on-body unit on user demand. According to such embodiments, the monitor unit 14 requests data from the on-body unit 11. Accordingly, embodiments include systems in which data is transferred from the on-body unit 11 to the monitor unit 14 only when the monitor unit 14 invokes data from the on-body unit 11.

The data transfer request may be provided in several ways. For example, in some embodiments, one or both of the on-body unit 11 and monitor unit 14 may include a mechanical switch activatable by a user or activated upon some other action or event, the activation of which initiates the transfer of signal from the on-body unit 11 to the monitor unit 14. A mechanical switch may be provided which is activatable by the user to cause power to be turned on to the on-body system, e.g., to electronic components that control the sensor.

In some embodiments, the monitor unit 14 is placed in close proximity (e.g., within a range of about one to about ten inches or less in certain embodiments) with the communications electronics of the on-body unit to cause initiation of data transfer from the on-body unit to the monitor unit, either over a wired connection, or wirelessly by various means, including, for example, various RF-carried encodings and protocols and IR links.

In some embodiments, a signal relating to analyte level is instantaneously generated by the analyte sensor 12 upon receipt of the request, and provided to the monitor unit 14 as requested.

In other embodiments, a signal relating to analyte level is obtained according to a schedule, e.g., once every 250 ms, once a second, once a minute, etc., and at least temporarily stored. Upon receipt of the data transfer request at the on-body unit 11 from the monitor unit 14, analyte-related information is provided to the monitor unit 14 from the on-body unit 11. In some cases, analyte-related information provided to the monitor unit 14 is or at least includes the most recent analyte signal(s).

In further embodiments, additional data is provided to the monitor unit 14 in response to or upon receipt of a request for such additional data. For example, analyte trend data may be provided. Such trend data may include two or more analyte data points to indicate that analyte levels are rising, falling, or stable. Analyte trend data may include data from longer periods of time, such as, e.g., several minutes, several hours, several days, or several weeks.

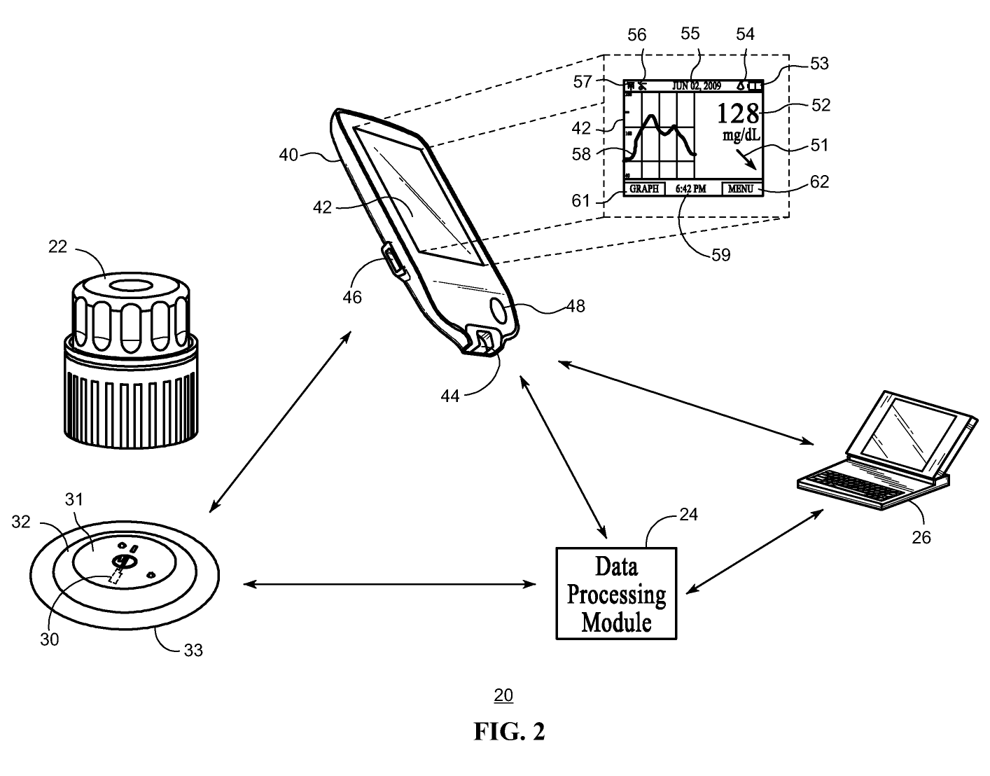

For purpose of illustration, and not limitation, an exemplary analyte monitoring system is depicted in FIG. 2. FIG. 2 shows an exemplary in vivo-based analyte monitoring system 20 in accordance with embodiments of the present disclosure. As shown, in certain embodiments, analyte monitoring system 20 includes sensor electronics 32 electrically coupled to in vivo analyte sensor 30, a proximal portion of which is shown in FIG. 2, and attached to adhesive layer 33 for attachment on a skin surface on the body of a user. Sensor electronics 32 includes on body housing 31, that defines an interior compartment. Also shown in FIG. 2 is insertion device 22 that, when operated, transcutaneously positions a portion of analyte sensor 30 through a skin surface and in fluid contact with ISF, and positions sensor electronics 32 and adhesive layer 33 on a skin surface In certain embodiments, sensor electronics 32, analyte sensor 30 and adhesive layer 33 are sealed within the housing of insertion device 22 before use, and in certain embodiments, adhesive layer 33 is also sealed within the housing or itself provides a terminal seal of the insertion device 22. Devices, systems and methods that maybe used with embodiments herein are described, e.g., in U.S. patent application Ser. Nos. 12/698,129, 12/873,133, 12/893,974, 12/895,015, 12/900,363, 13/071,461, 13/071,487, and 13/071,497, the disclosures of each of which are incorporated herein by reference for all purposes.

Referring back to the FIG. 2, analyte monitoring system 20 includes display device 40 which includes a display 42 to output information to the user, an input component 48 such as a button, actuator, a touch sensitive switch, a capacitive switch, pressure sensitive switch, jog wheel or the like, to input data or command to display device 40 or otherwise control the operation of display device 40. It is noted that some embodiments may include display-less devices or devices without any user interface components. These devices may be functionalized to store data as a data logger and/or provide a conduit to transfer data from sensor electronics and/or a display-less device to another device and/or location. Embodiments will be described herein as display devices for exemplary purposes which are in no way intended to limit the embodiments of the present disclosure. It will be apparent that display-less devices may also be used in certain embodiments.

In certain embodiments, sensor electronics 32 may be configured to store some or all of the monitored analyte related data received from analyte sensor 30 in a memory during the monitoring time period, and maintain it in memory until the usage period ends. In such embodiments, stored data is retrieved from sensor electronics 32 at the conclusion of the monitoring time period, for example, after removing analyte sensor 30 from the user by detaching sensor electronics 32 from the skin surface where it was positioned during the monitoring time period. In such data logging configurations, real time monitored analyte level is not communicated to display device 40 during the monitoring period or otherwise transmitted from sensor electronics 32, but rather, retrieved from sensor electronics 32 after the monitoring time period.

In certain embodiments, input component 48 of display device 40 may include a microphone and display device 40 may include software configured to analyze audio input received from the microphone, such that functions and operation of the display device 40 may be controlled by voice commands. In certain embodiments, an output component of display device 40 includes a speaker for outputting information as audible signals. Similar voice responsive components such as a speaker, microphone and software routines to generate, process and store voice driven signals may be provided to sensor electronics 32.

In certain embodiments, display 42 and input component 48 may be integrated into a single component, for example a display that can detect the presence and location of a physical contact touch upon the display such as a touch screen user interface. In such embodiments, the user may control the operation of display device 40 by utilizing a set of pre-programmed motion commands, including, but not limited to, single or double tapping the display, dragging a finger or instrument across the display, motioning multiple fingers or instruments toward one another, motioning multiple fingers or instruments away from one another, etc. In certain embodiments, a display includes a touch screen having areas of pixels with single or dual function capacitive elements that serve as LCD elements and touch sensors.

Display device 40 also includes data communication port 46 for wired data communication with external devices such as remote terminal (personal computer) 26, for example. Example embodiments of the data communication port 46 include USB port, mini USB port, RS-232 port, Ethernet port, Firewire port, or other similar data communication ports configured to connect to the compatible data cables. Display device 40 may also include an integrated in vitro glucose meter, including in vitro test strip port 44 to receive an in vitro glucose test strip for performing in vitro blood glucose measurements.

Referring still to FIG. 2, display 42 in certain embodiments is configured to display a variety of information—some or all of which may be displayed at the same or different time on display 42. In certain embodiments the displayed information is user-selectable so that a user can customize the information shown on a given display screen. Display 42 may include but is not limited to graphical display 58, for example, providing a graphical output of glucose values over a monitored time period (which may show important markers such as meals, exercise, sleep, heart rate, blood pressure, etc, numerical display 52, for example, providing monitored glucose values (acquired or received in response to the request for the information), and trend or directional arrow display 51 that indicates a rate of analyte change and/or a rate of the rate of analyte change, e.g., by moving locations on display 42.

As further shown in FIG. 2, display 42 may also include date display 55 providing for example, date information for the user, time of day information display 59 providing time of day information to the user, battery level indicator display 53 which graphically shows the condition of the battery (rechargeable or disposable) of the display device 40, sensor calibration status icon display 54 for example, in monitoring systems that require periodic, routine or a predetermined number of user calibration events, notifying the user that the analyte sensor calibration is necessary, audio/vibratory settings icon display 56 for displaying the status of the audio/vibratory output or alarm state, and wireless connectivity status icon display 57 that provides indication of wireless communication connection with other devices such as sensor electronics, data processing module 24, and/or remote terminal 26. As additionally shown in FIG. 2, display 42 may further include simulated touch screen button 61, 62 for accessing menus, changing display graph output configurations or otherwise for controlling the operation of display device 40.

Referring back to FIG. 2, in certain embodiments, display 42 of display device 40 may be additionally, or instead of visual display, configured to output alarms notifications such as alarm and/or alert notifications, glucose values etc, which may be audible, tactile, or any combination thereof. In one aspect, the display device 40 may include other output components such as a speaker, vibratory output component and the like to provide audible and/or vibratory output indication to the user in addition to the visual output indication provided on display 42. Further details and other display embodiments can be found in, e.g., U.S. patent application Ser. No. 12/871,901, and U.S. provisional application Nos. 61/238,672, 61/247,541, and 61/297,625, the disclosures of each of which are incorporated herein by reference for all purposes.

After the positioning of sensor electronics 32 on the skin surface and analyte sensor 30 in vivo to establish fluid contact with ISF (or other appropriate body fluid), sensor electronics 32 in certain embodiments is configured to wirelessly communicate analyte related data (such as, for example, data corresponding to monitored analyte level and/or monitored temperature data, and/or stored historical analyte related data) when sensor electronics 32 receives a command or request signal from display device 40. In certain embodiments, sensor electronics 32 may be configured to at least periodically broadcast real time data associated with monitored analyte level which is received by display device 40 when display device 40 is within communication range of the data broadcast from sensor electronics 32, i.e., it does not need a command or request from a display device to send information.

For example, display device 40 may be configured to transmit one or more commands to sensor electronics 32 to initiate data transfer, and in response, sensor electronics 32 may be configured to wirelessly transmit stored analyte related data collected during the monitoring time period to display device 40. Display device 40 may in turn be connected to a remote terminal 26 such as a personal computer and functions as a data conduit to transfer the stored analyte level information from the sensor electronics 32 to remote terminal 26. In certain embodiments, the received data from the sensor electronics 32 may be stored (permanently or temporarily) in one or more memory of the display device 40. In certain other embodiments, display device 40 is configured as a data conduit to pass the data received from sensor electronics 32 to remote terminal 26 that is connected to display device 40.

Referring still to FIG. 2, also shown in analyte monitoring system 20 are data processing module 24 and remote terminal 26. Remote terminal 26 may include a personal computer, a server terminal a laptop computer or other suitable data processing devices including software for data management and analysis and communication with the components in the analyte monitoring system 20. For example, remote terminal 26 may be connected to a local area network (LAN), a wide area network (WAN), or other data network for uni-directional or bi-directional data communication between remote terminal 26 and display device 40 and/or data processing module 24.

Remote terminal 26 in certain embodiments may include one or more computer terminals located at a physician's office or a hospital. For example, remote terminal 26 may be located at a location other than the location of display device 40. Remote terminal 26 and display device 40 could be in different rooms or different buildings. Remote terminal 26 and display device 40 could be at least about one mile apart, e.g., at least about 100 miles apart, e.g., at least about 1000 miles apart. For example, remote terminal 26 could be in the same city as display device 40, remote terminal 26 could be in a different city than display device 40, remote terminal 26 could be in the same state as display device 40, remote terminal 26 could be in a different state than display device 40, remote terminal 26 could be in the same country as display device 40, or remote terminal 26 could be in a different country than display device 40, for example.

In certain embodiments, a separate, optional data communication/processing device such as data processing module 24 may be provided in analyte monitoring system 20. Data processing module 24 may include components to communicate using one or more wireless communication protocols such as, for example, but not limited to, infrared (IR) protocol, Bluetooth® protocol, Zigbee protocol, and 802.11 wireless LAN protocol. Additional description of communication protocols including those based on Bluetooth® protocol and/or Zigbee protocol can be found in U.S. Patent Publication No. 2006/0193375 incorporated herein by reference for all purposes. Data processing module 24 may further include communication ports, drivers or connectors to establish wired communication with one or more of display device 40, sensor electronics 32, or remote terminal 26 including, for example, but not limited to USB connector and/or USB port, Ethernet connector and/or port, FireWire connector and/or port, or RS-232 port and/or connector.

In certain embodiments, data processing module 24 is programmed to transmit a polling or query signal to sensor electronics 32 at a predetermined time interval (e.g., once every minute, once every five minutes, or the like), and in response, receive the monitored analyte level information from sensor electronics 32. Data processing module 24 stores in its memory the received analyte level information, and/or relays or retransmits the received information to another device such as display device 40. More specifically in certain embodiments, data processing module 24 may be configured as a data relay device to retransmit or pass through the received analyte level data from sensor electronics 32 to display device 40 or a remote terminal (for example, over a data network such as a cellular or WiFi data network) or both.

In certain embodiments, sensor electronics 32 and data processing module 24 may be positioned on the skin surface of the user within a predetermined distance of each other (for example, about 1-12 inches, or about 1-10 inches, or about 1-7 inches, or about 1-5 inches) such that periodic communication between sensor electronics 32 and data processing module 24 is maintained. Alternatively, data processing module 24 may be worn on a belt or clothing item of the user, such that the desired distance for communication between the sensor electronics 32 and data processing module 24 for data communication is maintained. In a further aspect, the housing of data processing module 24 may be configured to couple to or engage with sensor electronics 32 such that the two devices are combined or integrated as a single assembly and positioned on the skin surface. In further embodiments, data processing module 24 is detachably engaged or connected to sensor electronics 32 providing additional modularity such that data processing module 24 may be optionally removed or reattached as desired.

Referring again to FIG. 2, in certain embodiments, data processing module 24 is programmed to transmit a command or signal to sensor electronics 32 at a predetermined time interval such as once every minute, or once every 5 minutes or once every 30 minutes or any other suitable or desired programmable time interval to request analyte related data from sensor electronics 32. When data processing module 24 receives the requested analyte related data, it stores the received data. In this manner, analyte monitoring system 20 may be configured to receive the continuously monitored analyte related information at the programmed or programmable time interval, which is stored and/or displayed to the user. The stored data in data processing module 24 may be subsequently provided or transmitted to display device 42, remote terminal 26 or the like for subsequent data analysis such as identifying frequency of periods of glycemic level excursions over the monitored time period, or the frequency of the alarm event occurrence during the monitored time period, for example, to improve therapy related decisions. Using this information, the doctor, healthcare provider or the user may adjust or recommend modification to the diet, daily habits and routines such as exercise, and the like.

In another embodiment, data processing module 24 transmits a command or signal to sensor electronics 32 to receive the analyte related data in response to a user activation of a switch provided on data processing module 24 or a user initiated command received from display device 40. In further embodiments, data processing module 24 is configured to transmit a command or signal to sensor electronics 32 in response to receiving a user initiated command only after a predetermined time interval has elapsed. For example, in certain embodiments, if the user does not initiate communication within a programmed time period, such as, for example about 5 hours from last communication (or 10 hours from the last communication, or 24 hours from the last communication), the data processing module 24 may be programmed to automatically transmit a request command or signal to sensor electronics 32. Alternatively, data processing module 24 may be programmed to activate an alarm to notify the user that a predetermined time period of time has elapsed since the last communication between the data processing module 24 and sensor electronics 32. In this manner, users or healthcare providers may program or configure data processing module 24 to provide certain compliance with analyte monitoring regimen, so that frequent determination of analyte levels is maintained or performed by the user.

In certain embodiments, when a programmed or programmable alarm condition is detected (for example, a detected glucose level monitored by analyte sensor 30 that is outside a predetermined acceptable range indicating a physiological condition which requires attention or intervention for medical treatment or analysis (for example, a hypoglycemic condition, a hyperglycemic condition, an impending hyperglycemic condition or an impending hypoglycemic condition), the one or more output indications may be generated by the control logic or processor of the sensor electronics 32 and output to the user on a user interface of sensor electronics 32 so that corrective action may be timely taken. In addition to or alternatively, if display device 40 is within communication range, the output indications or alarm data may be communicated to display device 40 whose processor, upon detection of the alarm data reception, controls the display 42 to output one or more notification.

In certain embodiments, control logic or microprocessors of sensor electronics 32 include software programs to determine future or anticipated analyte levels based on information obtained from analyte sensor 30, e.g., the current analyte level, the rate of change of the analyte level, the acceleration of the analyte level change, and/or analyte trend information determined based on stored monitored analyte data providing a historical trend or direction of analyte level fluctuation as function time during monitored time period. Predictive alarm parameters may be programmed or programmable in display device 40, or the sensor electronics 32, or both, and output to the user in advance of anticipating the user's analyte level reaching the future level. This provides the user an opportunity to take timely corrective action.

Information, such as variation or fluctuation of the monitored analyte level as a function of time over the monitored time period providing analyte trend information, for example, may be determined by one or more control logic or microprocessors of display device 40, data processing module 24, and/or remote terminal 26, and/or sensor electronics 32. Such information may be displayed as, for example, a graph (such as a line graph) to indicate to the user the current and/or historical and/or and predicted future analyte levels as measured and predicted by the analyte monitoring system 20. Such information may also be displayed as directional arrows (for example, see trend or directional arrow display 51) or other icon(s), e.g., the position of which on the screen relative to a reference point indicated whether the analyte level is increasing or decreasing as well as the acceleration or deceleration of the increase or decrease in analyte level. This information may be utilized by the user to determine any necessary corrective actions to ensure the analyte level remains within an acceptable and/or clinically safe range. Other visual indicators, including colors, flashing, fading, etc., as well as audio indicators including a change in pitch, volume, or tone of an audio output and/or vibratory or other tactile indicators may also be incorporated into the display of trend data as means of notifying the user of the current level and/or direction and/or rate of change of the monitored analyte level. For example, based on a determined rate of glucose change, programmed clinically significant glucose threshold levels (e.g., hyperglycemic and/or hypoglycemic levels), and current analyte level derived by an in vivo analyte sensor, the system 20 may include an algorithm stored on computer readable medium to determine the time it will take to reach a clinically significant level and will output notification in advance of reaching the clinically significant level, e.g., 30 minutes before a clinically significant level is anticipated, and/or 20 minutes, and/or 10 minutes, and/or 5 minutes, and/or 3 minutes, and/or 1 minute, and so on, with outputs increasing in intensity or the like.

Referring again back to FIG. 2, in certain embodiments, software algorithm(s) for execution by data processing module 24 may be stored in an external memory device such as an SD card, microSD card, compact flash card, XD card, Memory Stick card, Memory Stick Duo card, or USB memory stick/device including executable programs stored in such devices for execution upon connection to the respective one or more of the sensor electronics 32, remote terminal 26 or display device 40. In a further aspect, software algorithms for execution by data processing module 24 may be provided to a communication device such as a mobile telephone including, for example, WiFi or Internet enabled smart phones or personal digital assistants (PDAs) as a downloadable application for execution by the downloading communication device.

Examples of smart phones include Windows®, Android™, iPhone® operating system, Palm® WebOS™, Blackberry® operating system, or Symbian® operating system based mobile telephones with data network connectivity functionality for data communication over an internet connection and/or a local area network (LAN). PDAs as described above include, for example, portable electronic devices including one or more microprocessors and data communication capability with a user interface (e.g., display/output unit and/or input unit, and configured for performing data processing, data upload/download over the internet, for example. In such embodiments, remote terminal 26 may be configured to provide the executable application software to the one or more of the communication devices described above when communication between the remote terminal 26 and the devices are established.

In still further embodiments, executable software applications may be provided over-the-air (OTA) as an OTA download such that wired connection to remote terminal 26 is not necessary. For example, executable applications may be automatically downloaded as software download to the communication device, and depending upon the configuration of the communication device, installed on the device for use automatically, or based on user confirmation or acknowledgement on the communication device to execute the installation of the application. The OTA download and installation of software may include software applications and/or routines that are updates or upgrades to the existing functions or features of data processing module 24 and/or display device 40.

Referring back to remote terminal 26 of FIG. 2, in certain embodiments, new software and/or software updates such as software patches or fixes, firmware updates or software driver upgrades, among others, for display device 40 and/or sensor electronics 32 and/or data processing module 24 may be provided by remote terminal 26 when communication between the remote terminal 26 and display device 40 and/or data processing module 24 is established. For example, software upgrades, executable programming changes or modification for sensor electronics 32 may be received from remote terminal 26 by one or more of display device 40 or data processing module 24, and thereafter, provided to sensor electronics 32 to update its software or programmable functions. For example, in certain embodiments, software received and installed in sensor electronics 32 may include software bug fixes, modification to the previously stalled software parameters (modification to analyte related data storage time interval, resetting or adjusting time base or information of sensor electronics 32, modification to the transmitted data type, data transmission sequence, or data storage time period, among others). Additional details describing field upgradability of software of portable electronic devices, and data processing are provided in U.S. application Ser. Nos. 12/698,124, 12/794,721, 12/699,653, 12/699,844, 12/876,840 and 13/086,832, the disclosures of which are incorporated by reference herein for all purposes.

Further details regarding analyte monitoring systems are disclosed in U.S. Patent Publication Nos. 2009/0018425 A1, published Jan. 15, 2009; 2009/0054749 A1, published Feb. 26, 2009; 2009/0257911 A1, published Oct. 15, 2009, 2009/0281406 A1, published Nov. 12, 2009; 2009/0294277 A1, published Dec. 3, 2009; 2008/0058625 A1, published Mar. 6, 2008; 2008/0064937 A1, published Mar. 13, 2008; 2008/0071157 A1, published Mar. 20, 2008; 2008/0071158 A1, published Mar. 20, 2008; 2008/0179187 A1, published Jul. 31, 2008; 2008/0319295 A1, published Dec. 25, 2008; 2008/0319296 A1, published Dec. 25, 2008; 2007/0149873 A1, published Jun. 28, 2007; 2007/0149875 A1, published Jun. 28, 2007; 2009/0321277 A1, published Dec. 31, 2009; 2010/0030052 A1, published Feb. 4, 2010; and pending U.S. patent application Ser. Nos. 12/211,014, filed Sep. 15, 2008; 12/242,780, filed Sep. 30, 2008; 12/393,921, filed Feb. 27, 2009; 12/495,709, filed Jun. 30, 2009; 12/495,712, filed Jun. 30, 2009; 12/495,730, filed Jun. 30, 2009; 12/544,061, filed Aug. 19, 2009; 12/625,185, filed Nov. 24, 2009; 12/625,208, filed Nov. 24, 2009; 12/625,524, filed Nov. 24, 2009; 12/625,525, filed Nov. 24, 2009; 12/625,528, filed Nov. 24, 2009; 12/624,767, filed Nov. 24, 2009; 12/628,177, filed Nov. 30, 2009; 12/628,198, filed Nov. 30, 2009; 12/628,201, filed Nov. 30, 2009; 12/628,203, filed Nov. 30, 2009; 12/628,210, filed Nov. 30, 2009; 12/698,129, filed Feb. 1, 2010; 12/698,124, filed Feb. 1, 2010; 12/714,439, filed Feb. 26, 2010; 61/163,006, filed Mar. 24, 2009; 61/227,967, filed Jul. 23, 2009; 61/238,159, filed Aug. 29, 2009; 61/238,483, filed Aug. 31, 2009; 61/238,494, filed Aug. 31, 2009; 61/238,581, filed Aug. 31, 2009; 12/807,278, filed Aug. 31, 2010; 61/247,508, filed Sep. 30, 2009; 61/247,514, filed Sep. 30, 2009; 61/247,516, filed Sep. 30, 2009; 61/247,519, filed Sep. 30, 2009; 61/249,535, filed Oct. 7, 2009; 61/256,925, filed Oct. 30, 2009; 61/291,326, filed Dec. 31, 2009; 61/299,924, filed Jan. 29, 2010; 61/297,625, filed Jan. 22, 2010; 61/297,615, filed Jan. 22, 2010, each of which is incorporated by reference herein for all purposes.

### The Sensor

The analyte sensor, such as sensor 12 of FIG. 1 or sensor 30 of FIG. 2, of an analyte measurement system 10/20 may be used to monitor levels of a wide variety of analytes. Analytes that may be monitored include, for example, acetylcholine, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid-stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. One or more analyte may be monitored by a given sensor.

In embodiments of the present disclosure, sensor 12/30 is physically positioned in or on the body of a user whose analyte level is being monitored. Sensor 12/30 may be configured to continuously sample the analyte level of the user and convert the sampled analyte level, e.g., glucose concentration into a corresponding data signal, e.g., a current or voltage, for input into sensor electronics. The sensor electronics may amplify, filter, average, and/or otherwise process signals provided by the sensor.

The sensor may take on a number of forms. For example, the sensor may include a flexible or rigid substrate. In some embodiments, the sensor may be a wire. In some embodiments, the sensor may include two or three or more electrodes.

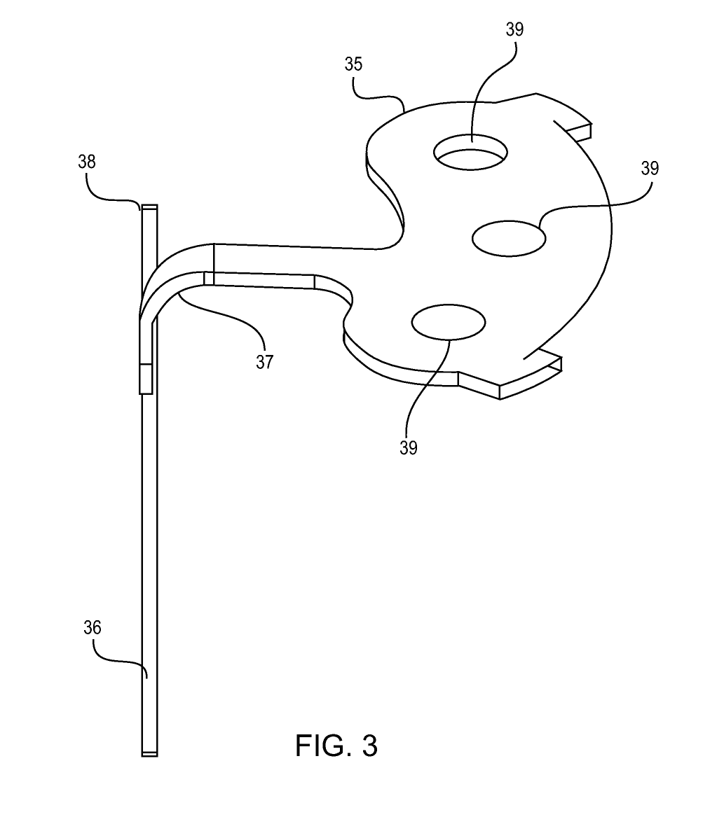

FIG. 3 is a view of an analyte sensor in accordance with certain embodiments of the present disclosure. The shape(s) described herein are exemplary only. Other sensor shapes are contemplated. In some embodiments, sensor 30 includes a substrate which is a dielectric, e.g., a polymer or plastic material, such as polyester or polyamide. In this embodiment, the sensor is constructed so that a distal portion 36 is positionable beneath skin and a proximal portion 35 is above skin. Accordingly, sensor 30 includes an insertion or internal distal portion 36 and an external or electrical contact proximal portion 35. The contact portion 35 typically includes several conductive contacts 39 (herein shown as three contacts) for connection to other electronics, e.g., at the sensor electronics 13 (FIG. 1) or sensor electronics 32 (FIG. 2). The contacts provided in this embodiment are for a working electrode, a reference electrode, and a counter electrode. In some embodiments, two or more working electrodes are provided. The operative portions of these electrodes, that is, working electrode, reference electrode, and counter electrode (not individually shown), are provided at the insertion portion, e.g., at the distal end of insertion portion 36. In some embodiments, one or more electrodes may be external to the body, e.g., an external counter electrode. The contact and operative portions of the electrodes are connected by circuit traces, which may run on the surface of the substrate. In some embodiments, the traces are provided in channels, or may be embedded within the substrate, or may traverse different sides of the substrate. In certain embodiments, the conductive traces may be provided on an internal layer of the sensor such that the traces are covered by one or more dielectric layers. The conductive contacts, conductive traces, and electrodes are fabricated from conductive material, such as platinum, palladium, gold, carbon, or the like. More than one material may be used for a given sensor. Further details of sensors are described, e.g., in U.S. Pat. Nos. 6,175,572 and 6,103,033, which are incorporated by reference herein for all purposes.

Sensor 30 may include a proximal retention portion 3. In some embodiments, the insertion portion 36 and the proximal retention portion 38 are substantially longitudinally aligned. The insertion portion 36 and the proximal retention portion 38 are sized and configured to be positioned with a sharp for installation into the skin of a subject, as described herein. In use, the sensor 30 may be configured to bend (as shown in FIG. 3) and therefore be positioned in two substantially perpendicular, intersecting planes.

Sensor 30 includes a laterally displaced portion (or sensor tab) and a connector portion, shown in this embodiments as a longitudinal displaced portion 37 which provides a path for electrical connections, e.g., the conductive traces, between the proximal 35 and distal 36 portions of the sensor 30. Sensor 30 is further provided, in certain embodiments, with a notch or cut-out between the proximal retention portion 38 and the longitudinal displaced portion 37. Such configuration facilitates the sensor 30 to bend and therefore be positioned in two substantially perpendicular, intersecting planes, as illustrated in FIG. 3. As will be described below, the sensor tab can be encased in the portion of the body of the sensor electronics to aid in securing and positioning the sensor 30. Proximal retention portion 38 maintains its longitudinal alignment with insertion portion 36 for positioning within an insertion sharp.

It is understood that sensor 30 may be positioned such that when the sensor is placed in contact with surrounding circuitry the contacts are downwardly positioned to engage circuitry below the contact portion 35 or, in some embodiments, the contacts are upwardly directed to engage circuitry positioned above the contact portion 35. In any of the embodiments of the sensor described herein, the distance between insertion portion 36 to retention portion 38 may be, e.g., about 5 mm, or about 10 mm, or about 15 mm, or about 20 mm.

Embodiments of analyte sensors have been described herein to operate electrochemically, through an arrangement of electrodes having chemical sensing layers applied thereto, by generating an electrical current proportional to the volume of a redox reaction of the analyte (and indicative of analyte concentration), catalyzed by an analyte-specific oxidizing enzyme. Embodiments exist in which the number of electrodes provided to bring about and detect the level of these reactions is two, three, or a greater number. However, other types of sensors may be employed as described herein.

A portion of sensor 30 may be situated above the surface of the skin, with a distal portion 36 penetrating through the skin and into the subcutaneous space in contact with the user's biofluid, such as interstitial fluid. Further details regarding the electrochemistry of sensor 30 is provided in U.S. Pat. Nos. 5,264,104; 5,356,786; 5,262,035; 5,320,725; and 6,990,366, each of which is incorporated by reference herein for all purposes.

In some embodiments, the sensor is implantable into a subject's body for a usage period (e.g., a minute or more, at least one day or more, about one to about 30 days or even longer, about three to about ten days, about three to about seven days, or in some embodiments, longer periods of up to several weeks) to contact and monitor an analyte present in a biological fluid. In this regard, the sensor can be disposed in a subject at a variety of sites (e.g., abdomen, upper arm, thigh, etc.), including intramuscularly, transcutaneously, intravascularly, or in a body cavity.

In some embodiments, sensor 30 is employed by insertion and/or implantation into a user's body for some usage period. In such embodiments, the substrate may be formed from a relatively flexible material.

While the embodiment illustrated in FIG. 3 has three electrodes, other embodiments can include a fewer or greater number of electrodes. For example, a two-electrode sensor can be utilized. The sensor 30 may be externally-powered and allow a current to pass proportional to the amount of analyte present. Alternatively, the sensor 30 itself may act as a current source in some embodiments. In some two-electrode embodiments, the sensor may be self-biasing and there may be no need for a reference electrode. An exemplary self-powered, two-electrode sensor is described in U.S. patent application Ser. No. 12/393,921, filed Feb. 26, 2009, and entitled “Self-Powered Analyte Sensor,” which is hereby incorporated by reference herein for all purposes. The level of current provided by a self-powered sensor may be low, for example, on the order of nanoamperes, in certain embodiments.

The mounting of the sensor 30 with respect to a housing, such as housing 31, is described in greater detail, e.g., in U.S. patent application Ser. Nos. 13/071,461, 13/071,487, 13/071,497, and 12/807,278, which are incorporated by reference in their entirety herein.

### Insertion Assembly

Insertion assemblies are provided, which are used to install a medical device to the subject. In some embodiments, an insertion assembly includes an inserter and the medical device itself. The inserter can be configured to insert various medical devices into the subject, such as for example, an analyte sensor, an infusion set, or a cannula. In some embodiments, the inserter can be configured to install a combination of such devices, e.g., a combined sensor/infusion set, etc., at the same or different times or locations. For example, in certain embodiments a given inserter can be configured to install a first device and a second device at different times. In this regard, the inserter can be reusable. For example, an inserter may be modifiable to be used with more than one medical device, to include more than one type of medical device, e.g., by attaching an adapter and/or removing detaching a portion of an inserter. The inserter can install the medical device in, under, or through the skin of the subject, or place the medical device on the surface of the skin. The medical device can include features or structures, e.g., barbs, tabs, adhesive, etc., to maintain the device in position with respect to the skin after insertion. The inserter device may also be used as a lancet, e.g., to pierce the skin without inserting or installing a medical device.

In some embodiments, an insertion assembly includes an inserter, a medical device, such as an analyte sensor, and a mount for supporting the medical device at least partially in or on the skin of the subject. In some embodiments, the mount is a support structure, plate and/or member which is attached, adhered, or otherwise secured to the skin of the subject. The mount may be inserted simultaneously with the medical device by the inserter. In other embodiments, the mount is installed after or before installation of the medical device. A mount may be applied by the inserter or separately. The mount may include features or structures (e.g., adhesive, guides, barbs, tabs, etc.) to maintain the sensor in position with respect to the skin after insertion and/or maintain the sensor in relative position with respect to the sensor electronics. In some embodiments, an adhesive pad or strip is used to secure the medical device, e.g., the sensor and/or the sensor electronics, and no mount is used.

In some embodiments, an insertion assembly includes an inserter, an analyte sensor, a mount, and a power supply. The mount and power supply may be inserted simultaneously with the analyte sensor by the inserter. In other embodiments, the mount and battery are installed after or before installation of the analyte sensor. In such case the mount and/or power supply may be applied by the inserter or separately. The power supply may be used to provide a current or a potential to the sensor and/or to provide power for communication of one or more signals to the monitor unit.

In some embodiments, an insertion assembly includes an inserter, a medical device such as an analyte sensor, a mount, and sensor electronics. The mount and sensor electronics may be deployed and/or installed simultaneously with the analyte sensor by the inserter. In other embodiments, the mount and sensor electronics are installed after or before installation of the analyte sensor. For example, the mount and the analyte sensor may be installed by the inserter, and the sensor electronics may be subsequently installed. In other embodiments, the mount is installed, followed by insertion of the analyte sensor by the inserter, and further followed by installation of the sensor electronics. In other embodiments, the mount and sensor electronics are installed first, and the analyte sensor is subsequently installed.

In some embodiments, electronics of the sensor electronics provide a voltage or current to the analyte sensor. In some embodiments, the electronics processes signals provided by the analyte sensor. In further embodiments, the electronics may include communication functionality for providing signal relating to signal provided by the analyte sensor to a further component, such as, e.g., a monitor unit, a computer, or other component. In some embodiments, communications circuitry, such as RFID antenna or communications circuitry is provided. The power supply may be used to power some or all of these functions. In some embodiments, power is provided from the monitor unit, e.g., via inductive coupling.

An inserter can include a plurality of different components. For example, an inserter may include one or more components for advancing a sharp towards the skin of the subject. The sensor and sensor electronics and/or mounting structure may be supported by a support structure, such as a carriage. A driver may be provided for advancing the sharp and/or the analyte sensor/support structure. In some embodiments, the actuator is directly or indirectly coupled to the sharp and/or support structure, such that manual force and speed applied by the user to the actuator is transferred to the sharp and/or support structure. In some embodiments, the applied force drives the sharp and/or support structure between a retracted position and an advanced position. In some embodiments, the sensor and sensor electronics and/or mounting structure is maintained in a retracted position prior to installation by contacting projections extending inwardly from a sheath. In accordance with this embodiment, the sensor and sensor electronics and/or mounting structure are temporarily maintained operatively between the support structure and the projections disposed on the interior wall of the sheath.

An inserter can also include one or more components for retracting the sharp, while allowing the analyte sensor and optional mount and/or electronics to remain on the subject. The components for retracting the sharp can include a retractor. It is understood that the retractor and the actuator may be the same structure or include some common components. In some embodiments, the retractor is directly or indirectly coupled to the sharp such that the manual force applied by the user is transferred from the retractor to the sharp to retract the sharp from the skin. In other embodiments, a drive assembly may be provided to retract the sharp. For example, the drive assembly may include a spring, motor, hydraulic piston, etc., to retract the sharp away from the skin of the subject. The drive assembly may also include a linear drive component.

In some embodiments, the retractor withdraws the sharp upon actuation by the user. In such cases, the user actuates the retractor when it is desired to withdraw the sharp. For example, the retractor may include a release switch. Upon activation of the release switch, the drive assembly, e.g., the spring or other driver refracts the sharp from the skin. In other embodiments, the retractor and the actuator include common components. After activating the actuator to advance the sharp and the analyte sensor, the user releases the actuator, which allows the drive assembly to withdraw the sharp from the skin.

In some embodiments, the retractor withdraws the sharp without further user interaction after actuation of insertion. For example, the inserter may include features or components which automatically retract the sharp upon advancement of the sharp and support structure by a predetermined amount. Inserter devices, in which no further action by the user is required to initiate withdrawal of the sharp after insertion, are referred to herein as having “automatic” withdrawal of the sharp.

### Inserter Devices

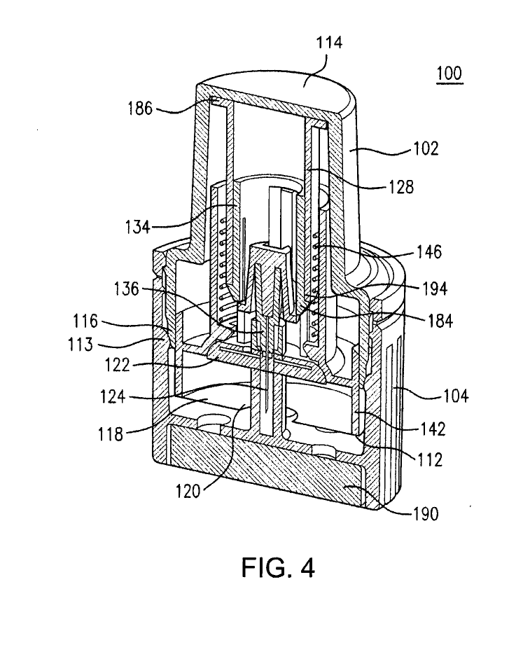

An inserter 100 in accordance with an exemplary embodiment is illustrated in FIG. 4. In some embodiments, inserter 100 has a maximum diameter of about 30 mm to about 60 mm, e.g., about 40 mm, about 43 mm, about 43.5 mm, about 50.5 mm, about 54.5 mm, etc. In some embodiments, inserter 100 has a maximum height of about 40 mm to about 80 mm, e.g., about 44 mm, about 46 mm, about 50 mm, about 53 mm, about 67 mm, about 71 mm, etc. In some embodiments, inserter 100 has a volume of about 35 cm³ to about 110 cm³, e.g., about 40 cm³, about 41 cm³, about 50 cm³, about 60 cm³, about 61 cm³, about 62 cm³, about 69 cm³, about 70 cm³, about 79 cm³, about 90 cm³, about 106 cm³, etc. Inserter 100 includes a handle 102 and a removable distal cap 104. The cap 104 may maintain a sterile, contaminant-free environment for the medical device and sharp housed therein. As illustrated in FIGS. 4-7, distal cap 104 is detachably secured to handle 102, e.g., by use of one or more mating members, e.g., threads 110 and 111, or hooks, tape, magnets, adhesive, friction-fit, snap-fit, and the like. Inserter 100 includes a base 142 which defines a distal surface 112, which may be a substantially planar surface as shown in this embodiment, for placement on the skin S of a subject, and in other embodiments may be a curved or inclined surface, e.g., a concave or convex surface. Inserter 100 may be utilized to advance a medical device into the skin of the subject, e.g., an analyte sensor, and infusion set, etc., as described herein. In some embodiments, handle 102 is advanced relative to base 142 in order to advance the medical device into the skin of the patient, as will be described in greater detail herein.

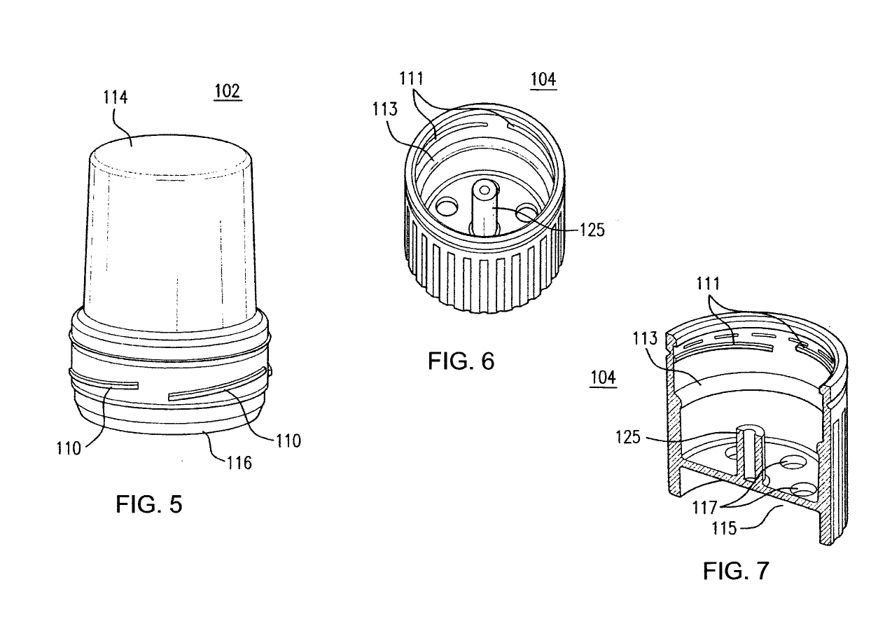

The components of inserter 100 are illustrated in FIGS. 5-21. As illustrated in FIG. 5, handle 102 includes a contact surface 114 for contact by a user to depress to advance the on body housing 122 (as illustrated in FIG. 4) and sensor 30 towards the skin of the subject. Threads 110 are provided on handle 102 (as illustrated in FIG. 5) for attachment to cap 104 via threads 111 (as illustrated in FIGS. 6-7). Cap 104 can include a receptacle, such as an upwardly extending sleeve 125 to assist holding on body housing 122 fixed in position. The distal portion of cap 104 includes a recess 115 for retaining a desiccant 190 therein. In some embodiments, a silica gel or molecular sieves may be used. Such material can be in granular form (pellets) or pressed into tablets, or otherwise. In some embodiments, silica gel tablets are used. Embodiments may include desiccant and/or packaging as described in U.S. patent application Ser. No. 12/714,439, which is incorporated by reference herein for all purposes.

Cap 104 may be provided with one or more apertures 117, which allows for passage of air to the desiccant 190 to remove moisture from the interior of the inserter 100. Cap 104 may include a stop such as an annular ridge 113, which engages the distal edge portion 116 of handle 102. In some embodiments, a stop such as annular ridge 113 prevents distal movement of handle 102 (as well as on body housing 122) when cap 104 is attached to handle 102.

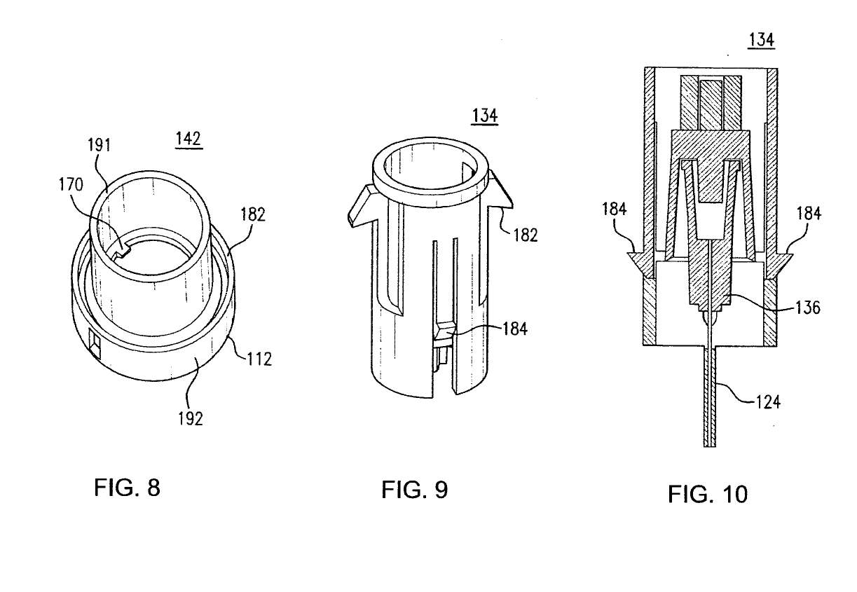

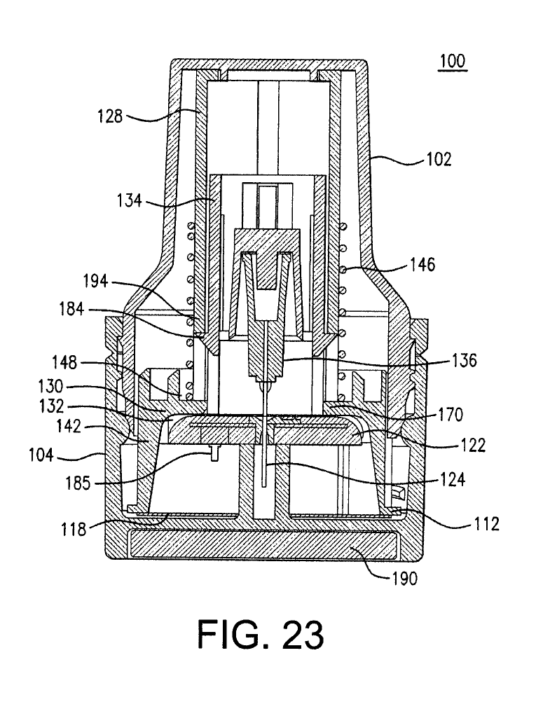

Base 142, as illustrated in FIGS. 4 and 8, includes a distal sheath portion 192, which shields sharp 124 and on body housing 122 prior to deployment and a distal rim 112 having a substantially planar surface configuration to rest on the subject's skin S. Base 142 also includes side walls 191, which along with inner rail 128 defines a recess for retraction spring 146. Base 142 provides a spring retention portion 148, as illustrated in FIG. 23.

Support member or shuttle 134, as illustrated in FIGS. 8-9, supports needle hub 136, from which sharp 124 extends longitudinally within the inserter 100. In some embodiments, the sharp is supported at an oblique angle, e.g., between and including about 0° (parallel to the skin) and about 90° (normal to the skin) with respect to the skin surface. Needle hub 136 can be secured to shuttle 134 via interlocking snaps or tabs, an O-ring configuration, adhesive, insert-molding, or other techniques known in the art. Alternatively, needle hub 136 may be integrally molded with shuttle 134. In some embodiments, sharp 124 is a solid needle. In some embodiments, sharp 124 is provided with a substantially cylindrical configuration defining an interior bore, e.g., a rigid cylindrical member or a hypodermic-style needle.

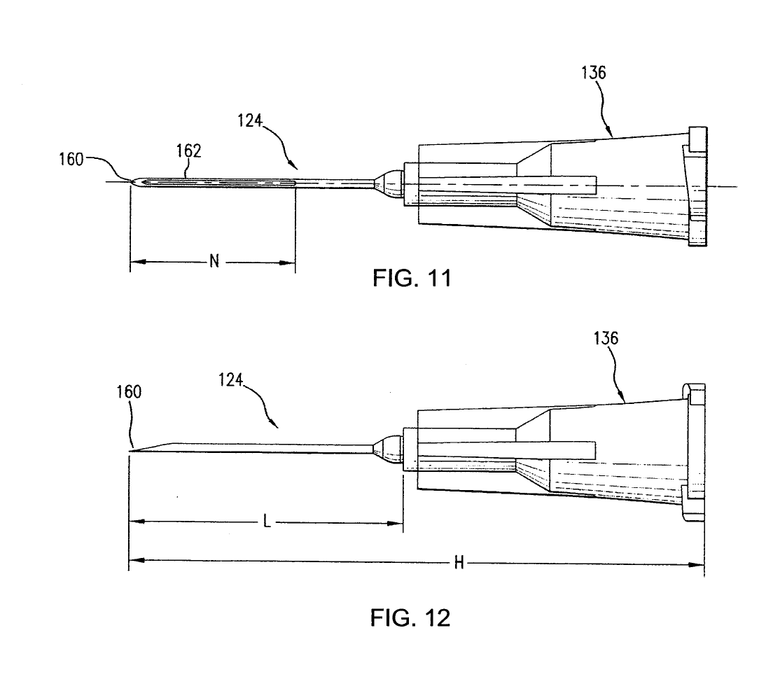

Needle hub 136 is further illustrated in FIGS. 11-12. Needle hub 136 supports sharp 124, having a sharpened distal portion 160. In some embodiments, as discussed herein, a longitudinal wall opening or gap 162 is provided in at least a portion of the wall of the sharp 124. The length N of the gap 162 is selected to be commensurate with the length of the insertion portion 36 through to the proximal retention portion 38 of the sensor, and in certain embodiments may be about 3 mm to about 50 mm, e.g., about 5 mm, or about 10 mm, or about 15 mm, or about 20 mm. The length L of the sharp 124 may be about 3 mm to about 50 mm, e.g., 5 mm or more, or about 10 mm, or about 20 mm, or about 30 mm, or about 50 mm, and is selected based upon the length of the insertion portion 36 of a sensor and the desired depth of the insertion portion 36 of the sensor 30. In some embodiments, the distance or spacing between the two edges of the gap is about 0.2 mm to about 0.5 mm, e.g., about 0.22 mm, about 0.25 mm, etc.

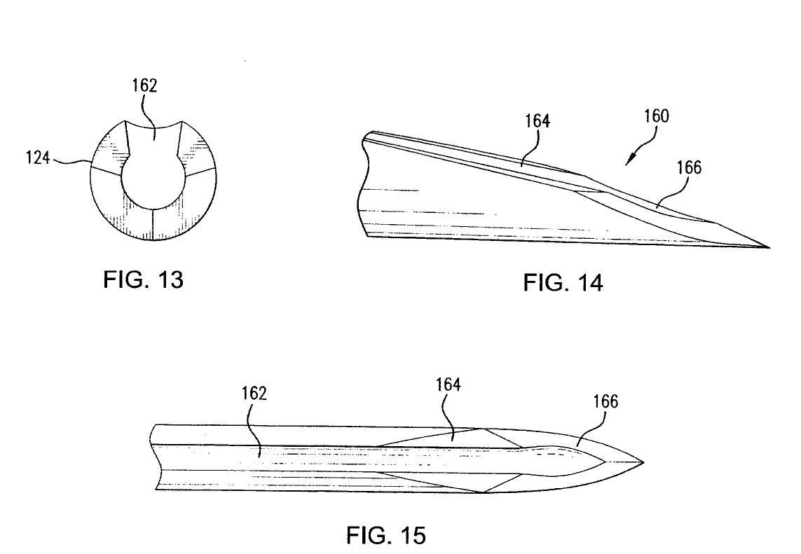

The distal portion 160 of sharp 124 is illustrated in greater detail in FIGS. 13-15. As illustrated in FIG. 13, sharp 124 has a substantially “C”- or “U”-shaped profile in this embodiment, but may have other configurations, e.g., substantially “V”-shaped. A longitudinal gap 162 is provided in the wall of the sharp 124. FIG. 14 illustrates distal portion 160 is provided with an angled tip. In some embodiments, the angled tip may be provided with a first angled tip portion 164 and a second steep-angled tip portion 166. The exemplary configuration, which includes multiple edges and faces, provides a sharp point to reduce penetration force, trauma, and bleeding for the subject. The distal section of the sensor body has a width sized to fit within the notch 162 of the insertion sharp 124 having a diameter less than about 20 to about 26 gauge, e.g., 21 gauge to about 25 gauge, where in certain embodiments the sharp is 21 gauge or 23 gauge or 25 gauge. Such sharp may be used with a sensor having a width or diameter—at least the portion that is carried by the sharp—of about 0.20 mm to about 0.80 mm, e.g., about 0.25 mm to about 0.60 mm, where in some embodiments the width or diameter of at least a portion of a sensor is 0.27 mm or 0.33 mm or 0.58 mm. In some embodiments, sharp 124 is fabricated from a sheet of metal and folded into a substantially “V” or “U” or “C” configuration in cross-section. Various technologies can be used to manufacture a folded sheet of metal to form sharp 124. For example, etched-sheet metal technology can be used to form the sharp 124. In this manner, the sharp can be formed having a very sharp edge so that penetration through the skin during insertion is less painful. In other embodiments, a progressive die technology may be utilized to form a complex sheet-metal shape that has a sharp edge. In some embodiments, the sharp 124 can be molded with a plastic cap so that the sharp can be handled during the inserter assembly process. Further, the die cut sharp may be molded with plastic to reinforce the “V,” “U,” or “C” shaped sheet metal configuration. In other embodiments, a laser-cut sharp can be formed. In this manner, the laser can be used to form the wall opening or gap 162 and first-angled tip portion 164 and a second, steep-angled tip portion 166.

In another embodiment, sharp 224 may be formed from a standard hypodermic needle. First, the hypodermic needle (having a circular cross-section) is cut to the desired length for sharp 224. Next, the hypodermic needle is compressed so that its cross-section is permanently deformed from a circular shape to an oval shape. The tip of the hypodermic needle is then ground to a bevel to produce a sharp point to reduce the required penetration force, as previously discussed. Finally, the top section of the needle is removed by appropriate techniques (e.g., grinding, electro polish, etc.). The resulting sharp 224 has a “U”-shaped configuration and provides ample space for the insertion of sensor 30. In some embodiments, the tip-grinding step and the compression step may be carried out in reversed order.

Due to the compression step, a user may initially start with a larger diameter hypodermic needle so that the finished sharp 224 will have similar dimensions to the previously described sharps.

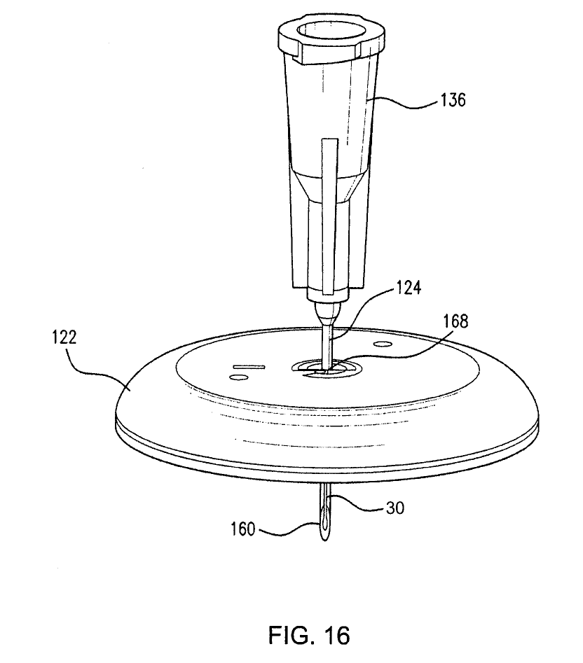

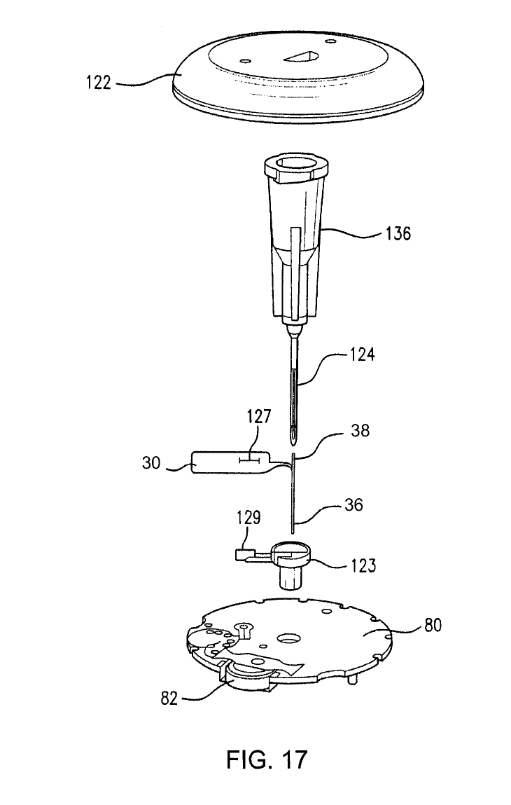

FIGS. 16-17 illustrate the position of on body housing 122 with respect to the needle hub 136 and sharp 124. The on body housing 122 can be configured to hold at least a portion of sensor 30 and sensor electronics. As illustrated in FIG. 16, the sharp 124 extends through an aperture 168 in the on body housing 122. Thus, in some embodiments, the sharp 124 is uncoupled to on body housing 122. The distal portion of sensor 30 is positioned with the sharp 124. As further illustrated in FIG. 17, electronics 80 of the sensor electronics 13/32 (e.g., a printed circuit board containing electronics components of the on-body unit 11) and sensor hub 123 are positioned within on body housing 122. Sensor 30 may include a positioning structure, or slit 127 which receives a positioning member, such as tab 129 of sensor hub 123. A power supply 82, such as a battery, e.g., a single use disposable battery, or rechargeable battery, is provided. The power supply 82 is used to provide potential or current to the sensor in some embodiments. In embodiments where a passive communications protocol such as passive RFID is used, no power supply is provided for the communications. Such power is provided by the monitor unit 14. In some embodiments where the sensor electronics 13/32 is used to transmit one or more signals, one or more power supplies may be used to provide power for such communications circuitry. In some embodiments, the active operational life of the battery may exceed the active operational life of the sensor 30.

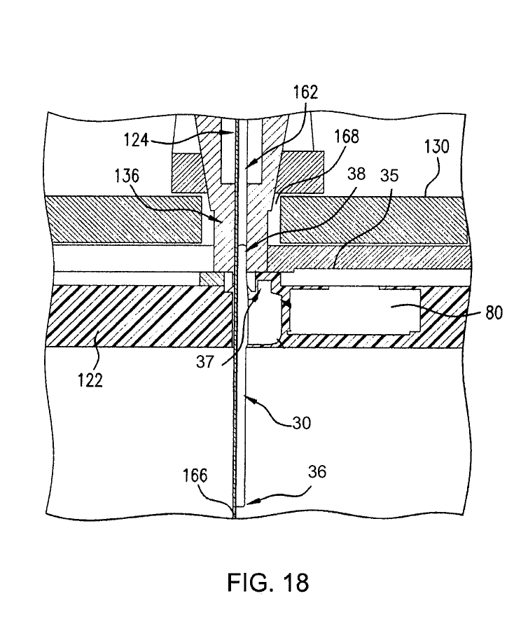

FIG. 18 illustrates in cross-section the orientation of the on body housing 122 with respect to the sharp 124 of inserter 100. As discussed herein, sensor 30 is disposed in a substantially bent configuration in some embodiments, such that a portion of the sensor, e.g., the insertion portion 36 and the proximal retention portion 38, are substantially vertical (e.g., substantially aligned with the longitudinal axis of the inserter 100 and substantially perpendicular to the skin surface) and the contact portion 35 (shown in profile) is oriented in a substantially horizontal configuration, and in electrical contact with the data-processing unit electronics, such as circuit 80. The sensor tab can be encased in the plastic of the on body housing 122 (e.g., “over molded”) and secured in place. The notch provides further stability to the sensor 30, e.g., by allowing the sensor tab to be encased by the material of the on body housing 122, and further provides a means for vertically orienting the sensor 30 during mounting, e.g., by allowing vertical positioning of the notch with respect to a vertical landmark of the on body housing 122.

The sensor 30, mounted with the on body housing 122, can be disposed within a recess of the carriage 130 such as a concave recess in the carriage 130. Alternatively, the sensor 30, mounted with the on body housing 122 can be disposed between the support structure and one or more projections extending from the wall of the sheath. In yet another alternative, the sensor 30 mounted with the on body housing 122 can be held in position by a releasable friction fit coupling to the sharp 124. In this manner, the carriage need not have a recess within which the sensor mounted with the sensor housing is disposed. In the initial configuration of the inserter 100 (see, e.g., FIG. 16) the sharp 124 extends through a longitudinal aperture 168 formed in a carriage 130. In some embodiments, the aperture 168 is appropriately sized, such that neither the sharp 124 nor needle hub 136 is in contact with the carriage 130. Accordingly, the needle hub 136 (and sharp 124) on the one hand, and the carriage 130 and the on body housing 122, on the other hand, move simultaneously but independently from one another. In other embodiments, a friction fit may be provided between the aperture and the sharp.

The insertion portion 36 and proximal retention portion 38 of the sensor 30 are disposed within a longitudinal bore 162 within the sharp 124 (See, e.g., FIG. 13). The proximal retention portion 38 is disposed within the longitudinal bore of the sharp 124 and provides additional stability to the mounting of the sensor 30 within the sharp 124. The longitudinal wall gap or opening 162 of sharp 124 is aligned with the sensor 30, such that the tab and the contact portion 35 extend laterally outward from the sharp 124.

In some embodiments, a resilient member may be included to provide frictional contact with the sharp 124 and/or the sensor 30. Such frictional contact provides additional stability between the on body housing 122 and sharp 124. In some embodiments, a resilient member may be formed as a spherical, ovoid, cylindrical, cube-shaped member, etc. Resilient member may be formed from any elastomeric material, e.g., molded plastic components, rubber, nitride, piton, urethane, etc.

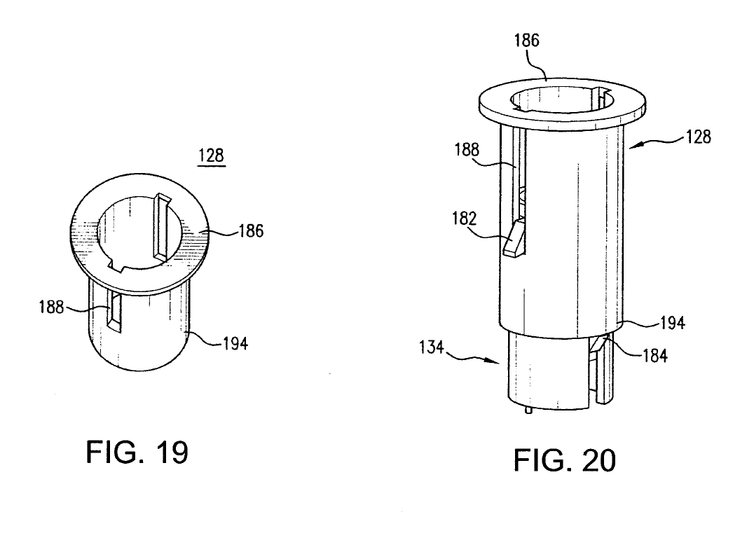

With continued reference to FIGS. 9 and 10, shuttle 134 includes wings 182 and resilient distally extending fingers 184. Inner rail 128 is illustrated in FIG. 19. As illustrated in FIG. 20, shuttle 134 is sized and configured for longitudinal movement within inner rail 128. Wings 182 of shuttle 134 are configured for longitudinal movement within axial notches 188 of inner rail 128. When fingers 184 of shuttle 134 are disposed in their normally biased outward position, fingers 184 engage the lower surface 194 of inner rail 128. In the configuration illustrated in FIG. 20, shuttle 134 is locked with respect to inner rail 128. As will be discussed herein, fingers 184 may be biased radically inward to allow upward movement of shuttle 134 relative to inner rail 128.

As illustrated in FIG. 4, inner rail 128 includes an upper surface 186 for engagement with handle 102. In some embodiments, surface 186 is adhered or otherwise fixed to handle 102.

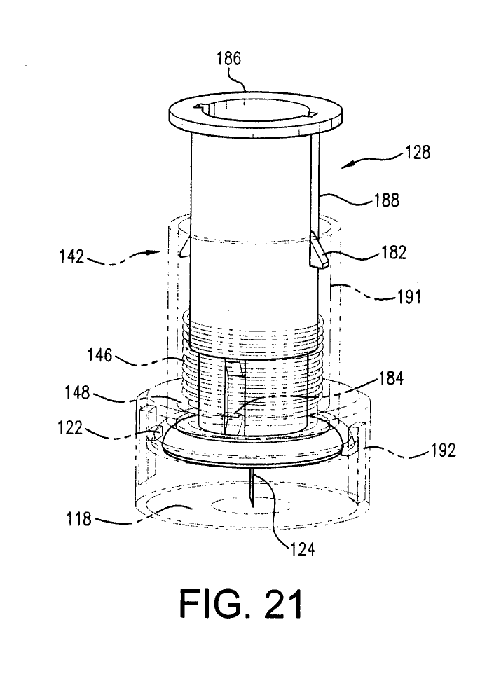

The relationship of inner rail 128, shuttle 134 and base 142 is illustrated in FIG. 21. In an initial configuration, inner rail 128 and shuttle 134 are in a locked relationship by engagement of wings 182 and fingers 184. Inner rail 128 and shuttle 134 are axially movable within base 142. Spring 146, which is secured between spring retention portion 148 of base 142 and wings 182 of shuttle 134 biases the inner rail 128 and shuttle 134 in a proximal (upward) direction.

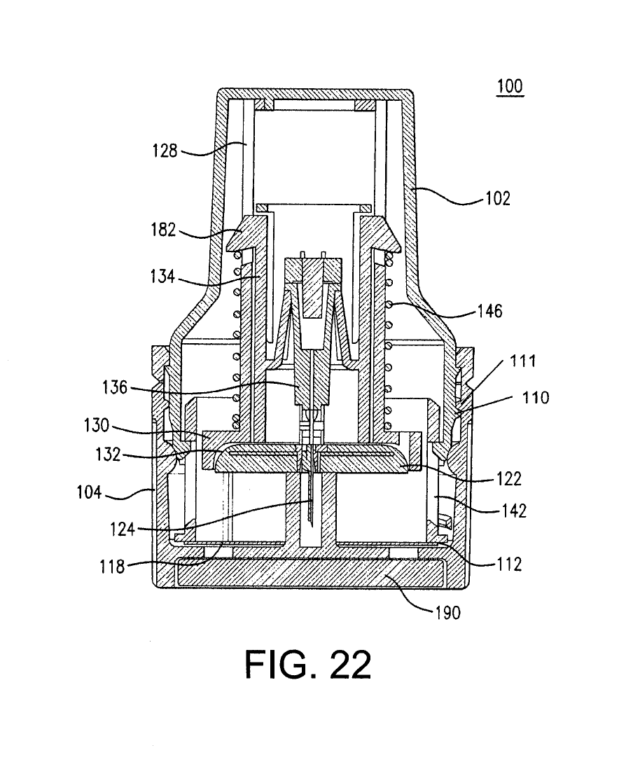

Inserter 100 is illustrated in section in FIGS. 22-23 prior to use in a sensor redeployment position. Cap 104 is attached to the distal portion of inserter 100, e.g., via inter-engagement of threads 110 and 111.

As illustrated in FIG. 22, the inserter 100 includes an initial configuration in which the handle 102 is disposed in a proximal position with respect to the base 142. In such configuration, the sharp 124 is disposed in a configuration spaced apart from an aperture of the adhesive layer 118.

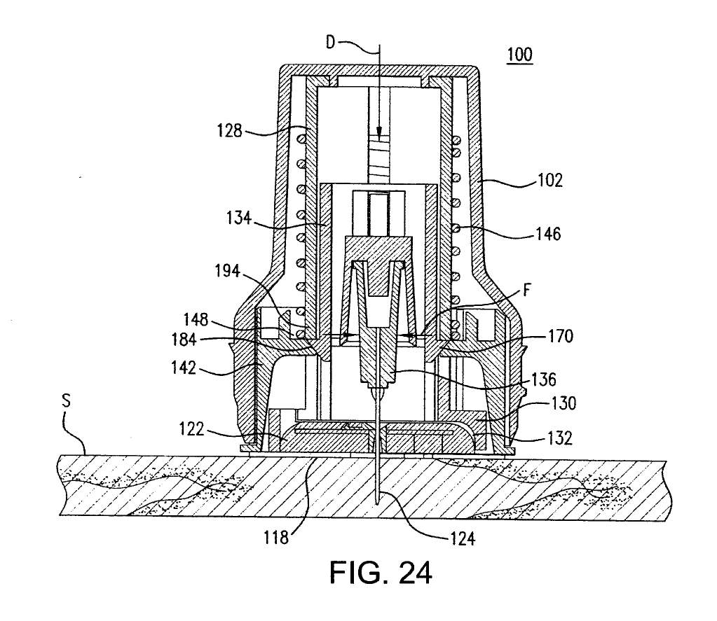

As illustrated in FIG. 24, inner rail 128 includes a carriage 130. In a sensor insertion position, the handle 102 is depressed downward (arrow D) against the bias of spring 146, the inner rail 128 moves downwardly with the carriage 130 and the on body housing 122. Shuttle 134 supports needle hub 136, from which sharp 124 extends longitudinally within the inserter 100. Initially, shuttle 134 is coupled to inner rail 128 via inter-engagement of fingers 184 of shuttle 134 with distal surface 194 of inner rail 128, and both shuttle 134 and inner rail 128 move distally together as a unit.

As the sharp 124 is urged distally towards the subject's skin (FIG. 24), it carries the sensor insertion portion 36 of sensor 30 into the subcutaneous portion of the subject's skin S and into contact with the interstitial fluid. As carriage 130 reaches a distal position, the distal surface of the on body housing 122 engages the upper surface of adhesive pad 118, thereby becoming adhered to the skin surface S of the subject.

In some embodiments, components are provided which allow the shuttle to be disengaged from the handle 102. For example, flanges 170 on base 142 engage fingers 184 of shuttle 134. Fingers 184 are pivoted or bend inwards by contact with flanges 170 (as indicated by arrows F).

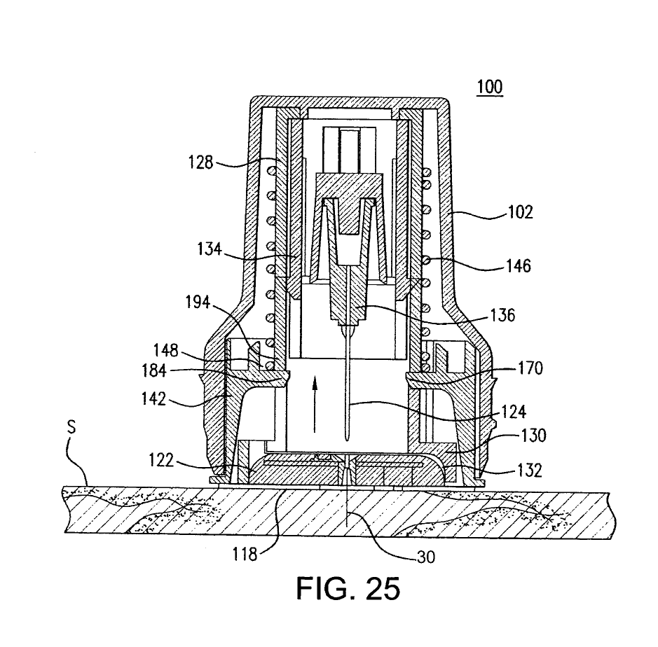

As illustrated in FIG. 25, such pivoting of fingers 184 causes fingers 184 to become disengaged from distal edge 194 of inner rail 128. Shuttle 134 is thereby disengaged from inner rail 128. Disengagement of the shuttle 134 from the inner rail 128 permits the spring 146 decompress and to expand, thereby advancing the shuttle 134 to a proximal or retracted position, and withdrawing the sharp 124 from the on body housing 122, withdrawing from its position around sensor 30, and receding from skin S of the subject, while leaving the sensor 30 in the skin and the on body unit 16 attached to the skin surface by adhesive pad 118. Once the sharp has been withdrawn from the subject, it is no longer accessible from the distal portion of the inserter 100, which prevents accidental needle sticks. When the carriage 130 reaches the distal position in which flanges 170 engage fingers 184 of needle shuttle 134, needle shuttle 134 withdraws sharp 124 automatically without further input from the user.

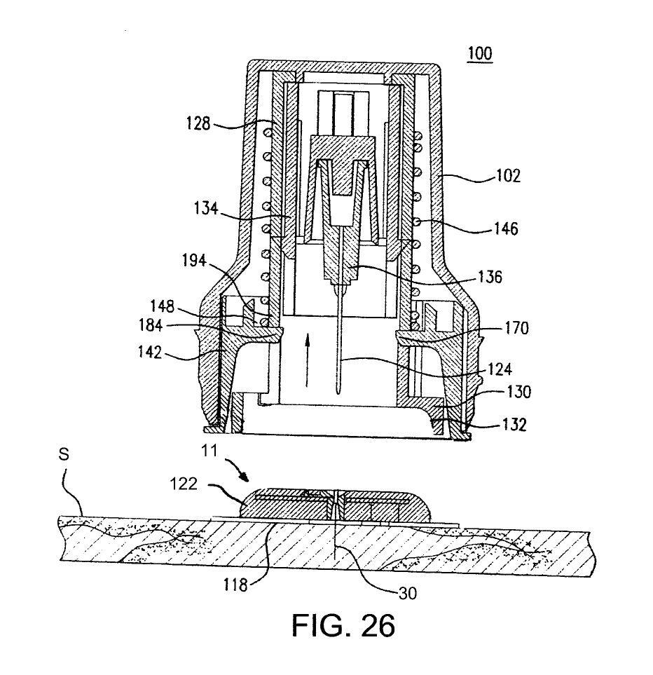

As illustrated in FIG. 26, the inserter 100 may be removed from the skin S of the subject, leaving the on-body unit 11 installed on the subject, e.g., lower surface of housing 122 adhered to the skin S via adhesive pad 118 and sensor 30 at least partially inserted in the skin and in contact with the interstitial fluid.

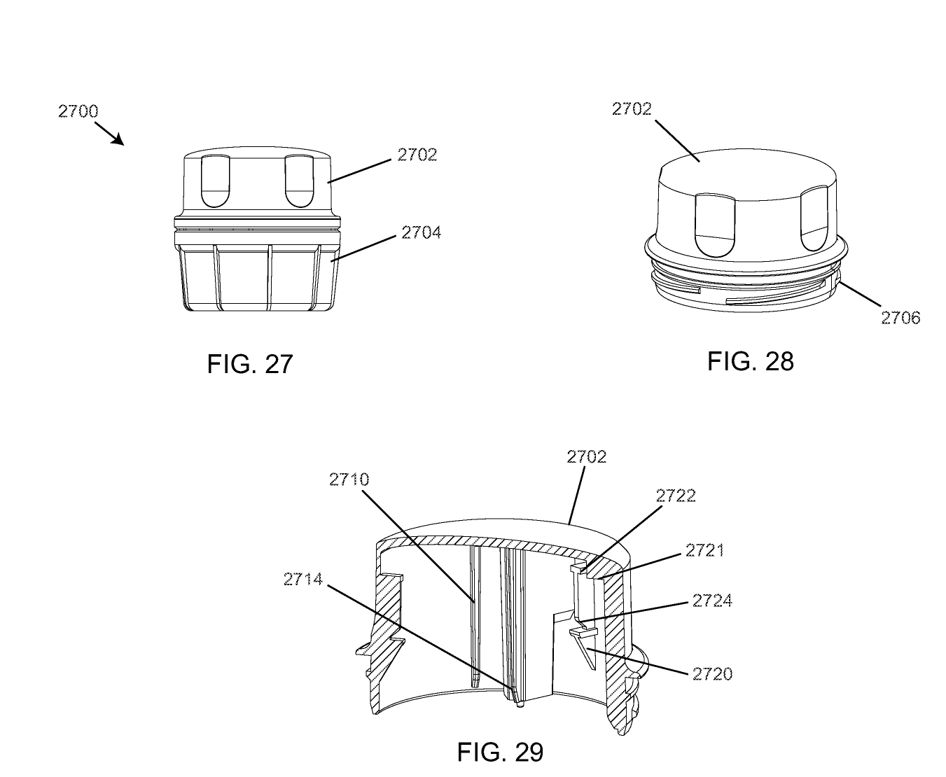

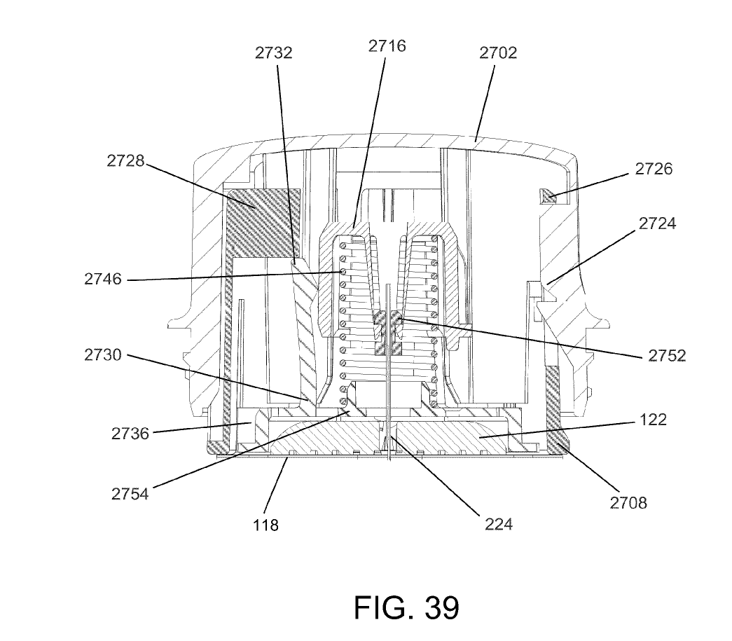

A further embodiment of an inserter is illustrated in FIGS. 27-41, and designated inserter 2700. In some embodiments, inserter 2700 has a maximum diameter of about 30 mm to about 60 mm, e.g., about 40 mm, about 43 mm, about 43.5 mm, about 46 mm, about 50 mm, etc. In some embodiments, inserter 2700 has a maximum height of about 40 mm to about 80 mm, e.g., about 44 mm, about 46 mm, about 49.5 mm, about 55 mm, about 67 mm, about 71 mm, etc. In some embodiments, inserter 2700 has a volume of about 35 cm³ to about 110 cm³, e.g., about 40 cm³, about 41 cm³, about 50 cm³, about 60 cm³, about 61 cm³, about 62 cm³, about 69 cm³, about 70 cm³, about 79 cm³, about 90 cm³, about 106 cm³, etc.

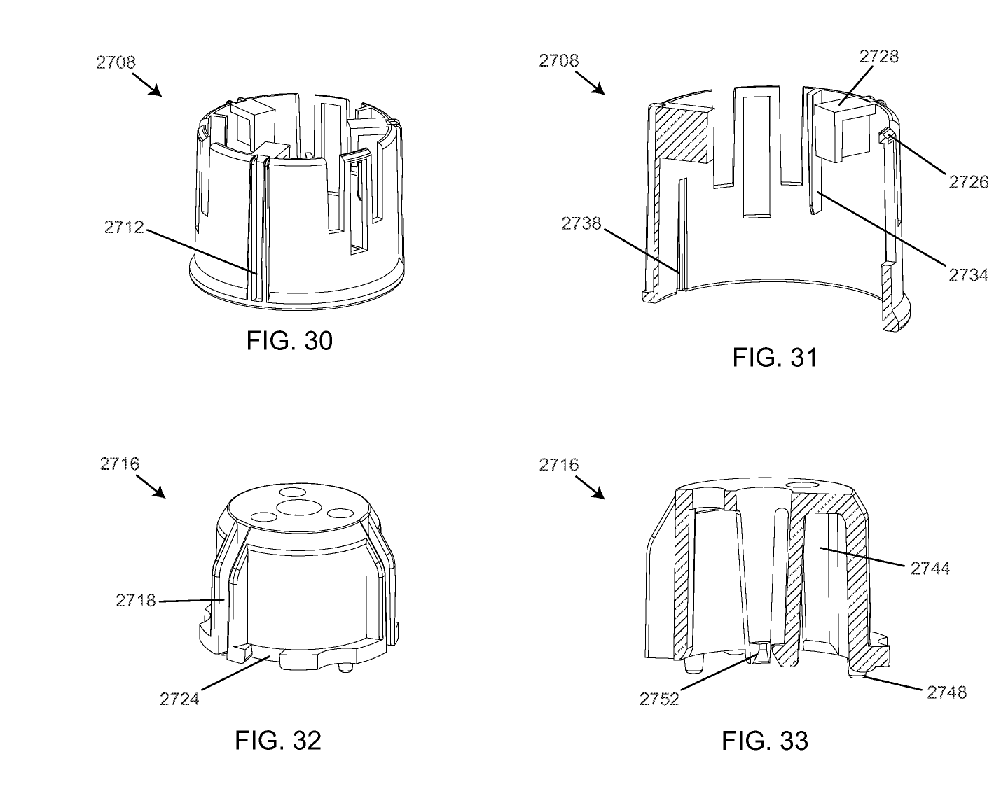

Inserter 2700 generally includes, e.g., a housing 2702 (FIGS. 28-29), sheath 2708 (FIGS. 30-31), and a removable distal cap 2704 for maintaining a sterile environment for the medical device and sharp housed therein (FIG. 27). As illustrated in FIGS. 28-29, housing 2702 is shown removed from distal cap 2704. Distal cap 2704 is detachably secured to housing 2702, e.g., by use of threads 2706. It is understood that cap may be secured using snap-fit or press-fit configuration. Inserter 2700 may be utilized to advance a medical device into the skin of the subject. Sheath 2708 generally a cavity or open space, within which sharp carrier 2716 and medical device carrier 2730 are moveable. In some embodiments, housing 2702 is advanced relative to sheath 2708 in order to advance the medical device distally and into the skin of the patient.

Housing 2702 includes sheath guide rail 2710 which interfaces with rail guides 2712 located on sheath 2708 (FIG. 30), thereby allowing housing 2702 to slidingly move relative to sheath 2708. Housing 2702 may further includes sharp carrier guide rail 2714 which interfaces with rail guides 2718 located on sharp carrier 2716 (FIG. 33). Sheath 2708, sharp carrier 2716, and housing 2702 may alternatively move relative to one another without the use of guide rails.

Ledge 2720 and/or ledge 2722 are provided on an interior portion of housing 2702. Ledge 2720 engages sheath 2708 to hold sheath 2708 in a pre-use position prior to insertion of the medical device. Ledge 2722 engages sheath 2708 to secure sheath 2708 in a post-use position after insertion of the medical device. Housing 2702 further includes detent 2724 which prevents housing 2702 from moving relative to sheath 2708 until a minimum force has been applied, e.g., distally by user to housing 2702. In some embodiments, housing 2702 includes insertion hard stop 2724. The sheath 2708 is secured to the housing 2702 via snap 2726. Snap 2726 snaps into the housing detent 2724. (In some embodiments, it is pinched between ledge 2720 and detent 2724, thus controlling its height relative to the housing 2702). The needle carrier 2716 is located and secured to the medical device carrier 2730 (located via interaction of locating features 2748 and 2750 and secured via interaction of carrier arms 2732 and angled top surface of 2716). The insertion hard stop 2724 is a controlled surface onto which the top of sheath surface 2728 will engage at the end of the insertion stroke to prevent further relative movement in some embodiments.

Further components of inserter 2700 are illustrated in FIGS. 30-35. Sheath 2708 is a generally cylindrical component. As illustrated in FIGS. 30-31, sheath 2708 may include attachment snaps 2726 which are biased into detent 2724 of housing 2702 to create a minimum force that must be overcome in order to advance sharp 224 into the subject's skin and install the on body housing 122. In some embodiments, the force to be overcome can be about 0.5 lbf to about 5 lbf., e.g., about 1 lbf, about 2 lbf, about 3 lbf, about 4 lbf, etc. Support wall 2728 prevents carrier arms 2732 on carrier 2730 from bending outwardly, clear of sharp carrier 2716. Ribs 2734 pinch carrier arms 2732 on carrier 2730, thus preventing on body housing 122 from falling out of inserter 2700 when sheath 2708 is in the extended position. Ribs 2734 are not present at the bottom of sheath 2708, thus allowing room for spring arms 2736 on carrier 2730 to release on body housing 122 when carrier 2730 has traveled to the bottom of sheath 2708. Slot 2738, located on sheath 2708, interfaces with locating feature 2740 on carrier 2730, thus orienting carrier 2730 to sheath 2708 during assembly.

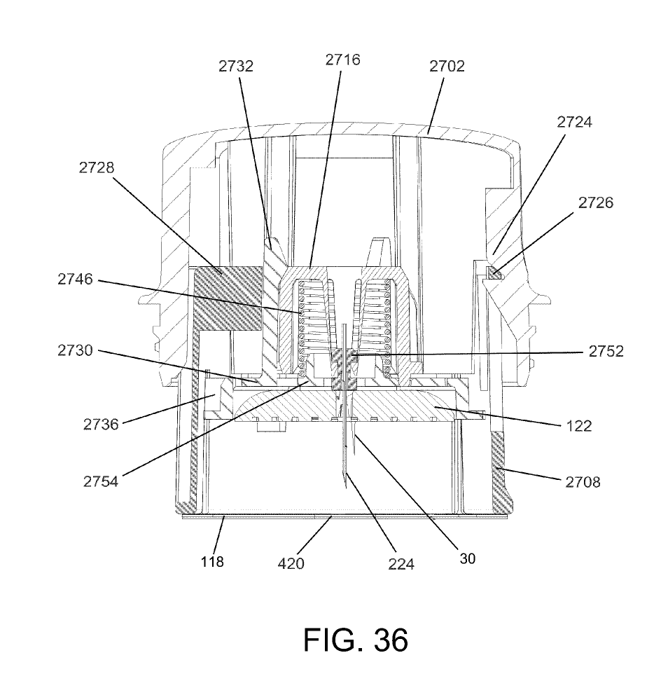

Referring next to FIGS. 32-33, depicted is sharp carrier 2716 in a perspective and cross-sectional view, respectively. Sharp carrier 2716 contains notches 2724 which allow clearance for the passage of carrier arms 2732 located on medical device carrier 2730. Guidance walls 2744 securely hold spring 2746 in place (FIG. 36). Locating features 2748, e.g., bosses or tabs, align with locating features 2750, e.g., recesses or apertures, on carrier 2730. Snap features 2752 secure sharp 224 securely within inserter 2700. It is contemplated that sharp 224 may be secured to sharp carrier 2716 by other techniques, e.g., friction fit, adhesive, welding, etc.

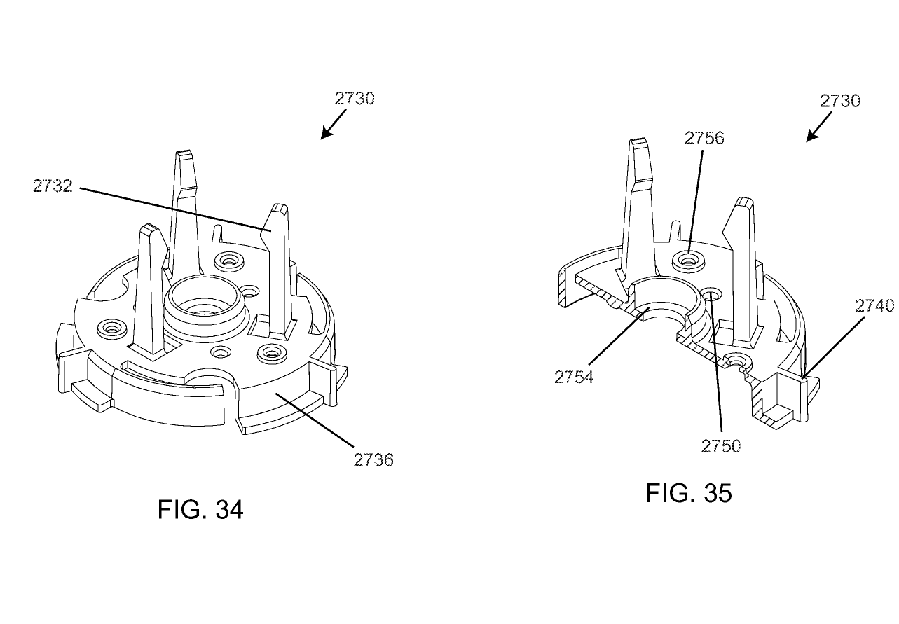

Medical device carrier 2730 is depicted in more detail in FIGS. 34-35. As shown, carrier 2730 contains spring locating ring 2754 which receives one end of spring 2746. In some embodiments, spring 2746 surrounds spring locating ring 2754. In some embodiments, the inner area remains clear to leave room for the deflection of sharp carrier feature snaps 2752 that deflect out when the sharp is inserted. Carrier 2730 further comprises locating features 2756 which interface with locating features on housing 2702.

Inserter 2700 is illustrated in cross-section in FIG. 36 in a state prior to use in which housing 2702 is disposed in a proximal position with respect to the sheath 2708. In such orientation, sharp 224 is disposed in a configuration spaced apart from the aperture 420 of the adhesive layer 118. The upper surface of spring 2746 is retained in inserter 2700 by sharp carrier 2716. The bottom surface of spring 2746 is retained by spring location ring 2754. Initially, spring 2746 is in a compressed or semi-compressed state while housing 2702 is disposed proximally from sheath 2708.

Sharp 224 extends longitudinally from sharp carrier 2716 within inserter 2700. In some embodiments, sharp 224 is supported at an oblique angle, e.g., between and including about 0° and 90° with respect to the skin surface.

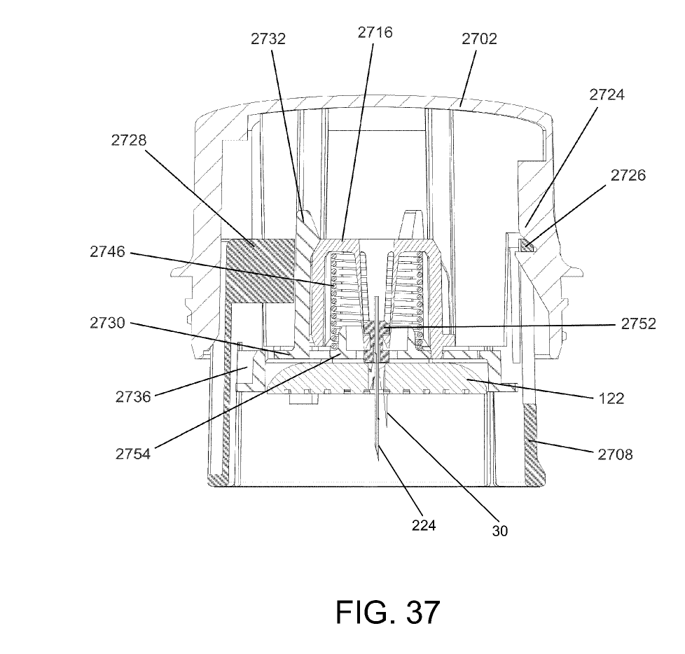

FIG. 37 illustrates inserter 2700 in cross-section after a user applies an initial downward force to housing 2702. In some embodiments, a predetermined minimum force must be used so that attachment snaps 2726 advance past detent 2724.

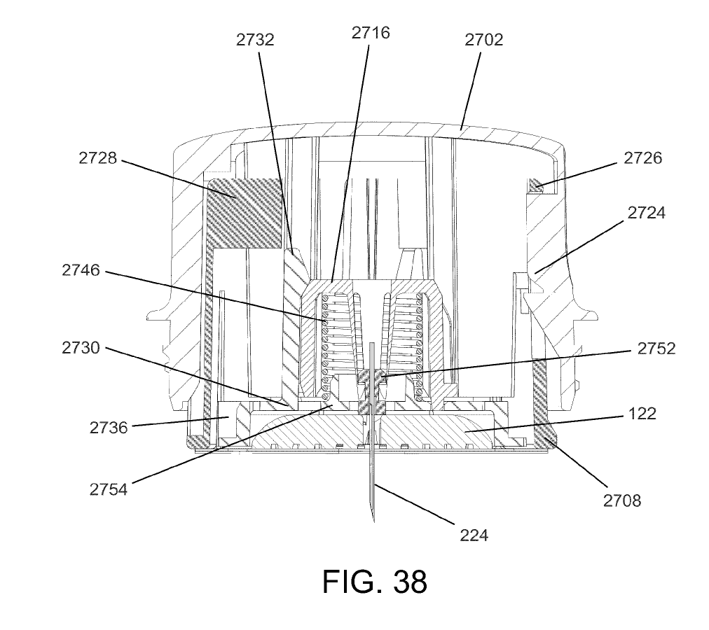

After detent 2724 has been overcome, e.g., snap 2726 is radially displaced, further depression of housing 2702 with respect to sheath 2708 causes distal longitudinal movement of the carrier 2730 and sharp carrier 2716, from a proximal position towards a distal position as shown in FIG. 38. As sharp 224 is further urged distally, it carries the sensor insertion portion 36 of sensor 30 (FIG. 38) into the subcutaneous portion of the subject's skin S.

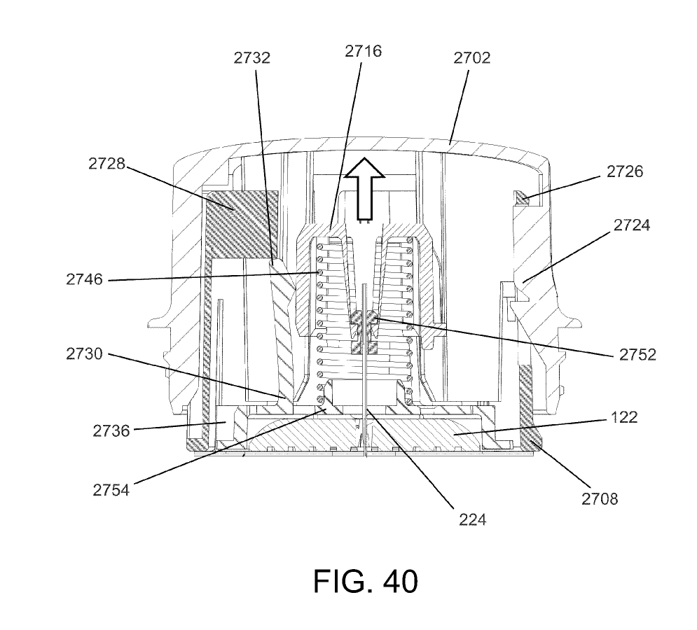

As carrier 2716 reaches a distal position, the distal surface of the on body housing 122 engages the upper surface of adhesive pad 118, thereby becoming adhered to the skin surface S of the subject. Concurrently, carrier arms 2732 are advanced distally and clear the support wall 2728. This allows carrier arms 2732 to deflect radially outwardly. When carrier arms 2732 deflect outwardly, shoulder portions of carrier arms 2732 are no longer in an interference relationship with the sharp carrier 2716. Thus spring 2746 is permitted to expand as shown in FIG. 40, thereby advancing the sharp carrier 2716 to a proximal position and withdrawing the sharp 224 from the skin S of the subject while leaving the on body housing 122 attached to the skin. Handle 2702 is maintained in the distal position. Sheath snap 2726 of the sheath 2708 have now moved up to lock over feature 2722 of the housing 2702. Now the housing 2702 and the sheath 2708 can no longer move longitudinally with respect to each other.

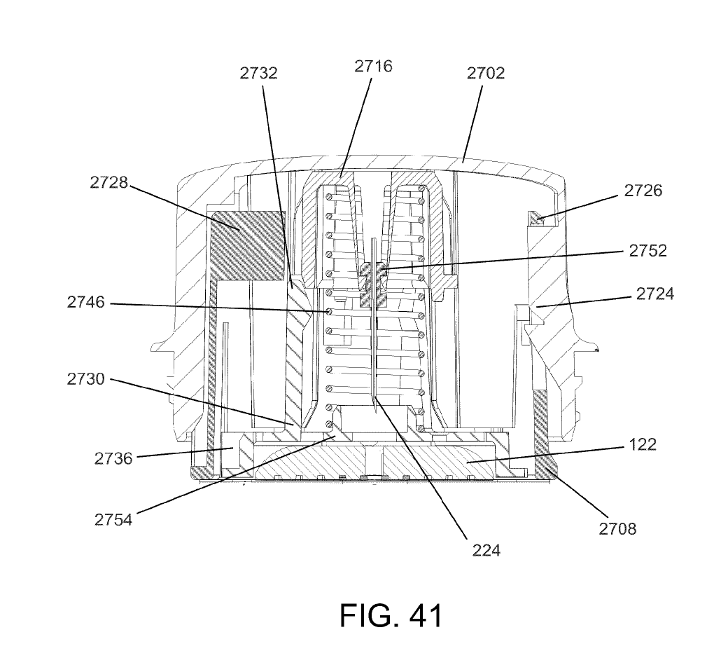

In some embodiments, the changing interaction of sheath snap 2726 with the housing detent/ledges 2720, 2724, and 2722 determine whether the sheath 2708 is locked. When snap 2726 is in the pre-fire position, ledge 2720 prevents sheath 2708 from being pulled out of the housing 2702. In this position, detent 2724 may also impede the movement of pushing the sheath 2708 into the housing 2702. When the detent is overcome by a at least at minimum force, the sheath 2704 moves longitudinally with respect to the housing 2702 until the snap 2726 snaps over housing ledge 2722. At this point, ledge 2722 prevents the sheath 2708 from being pulled out of the housing again, but from a new position (this position may be referred to as the used/post-fire position). Sharp carrier snap 2752 function is to hold onto the sharp/needle. In some embodiments, the sharp/needle carrier 2716 is held in the post-fire position relative to the housing 2702 by, e.g., an interference between the rails of the housing 2714 and the guide rails of the sharp carrier 2718 (this interference is only present once the sharp carrier is fully retracted) and/or by medical device carrier projections 2732 interfering with the bottom/floor of the sharp carrier (See, e.g., FIG. 41).

### On Body Housing

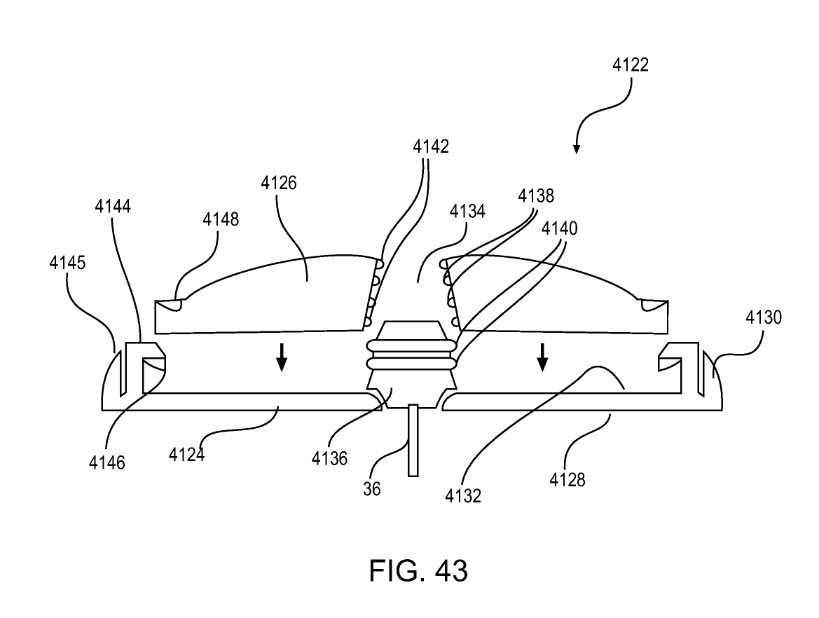

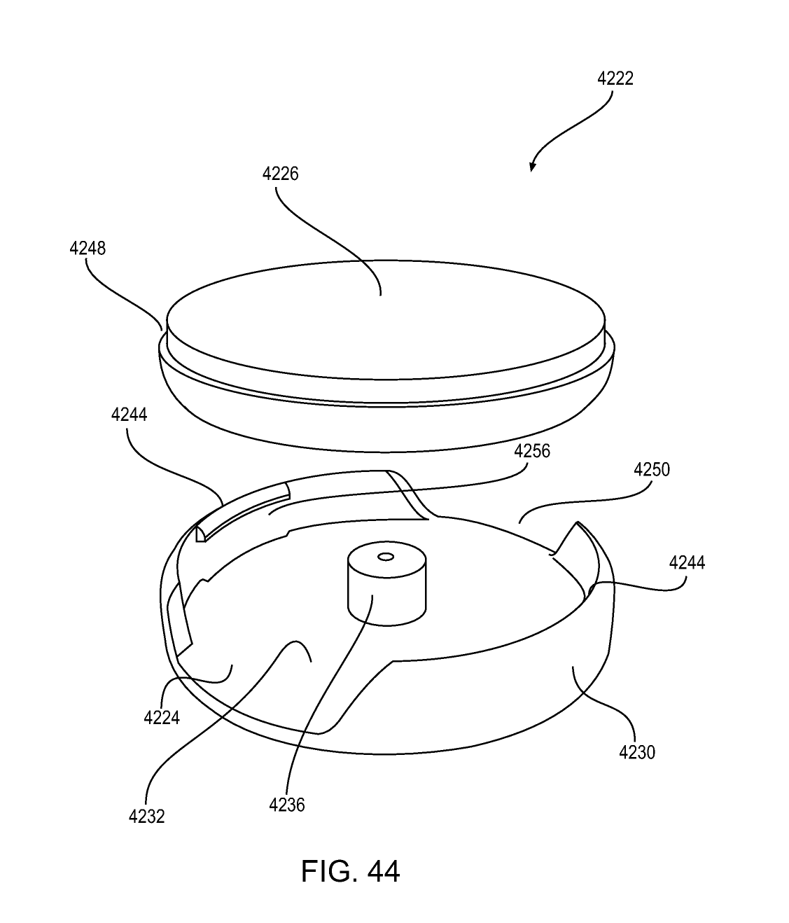

Another embodiment of the on body housing, referred to as on body housing 31 or 122 hereinabove, is illustrated in FIGS. 43-44. On body housing 4122 may be provided with a substantially circular configuration, or other shape, such as an elliptical or “football” shape as illustrated in FIG. 43. As with on body housing 122, on body housing 4122 has a reduced height (i.e., “Z”-dimension) to provide a low profile when sitting on the skin of the subject. In some embodiments, the height is about 3 mm to about 25 mm, e.g., may be about 4 mm, about 5 mm, about 10 mm, or about 15 mm. In certain embodiments, the on body housing 4122 may have a variable height such that it may have at least one portion that has a height that differs from at least one other portion. Electronics 80, for example, may include one or more of, e.g., an analog interface for connecting to the sensor 30, a processor, which may include, e.g., an ASIC, antenna, power supply to power the sensor and/or communications components, and capacitors.

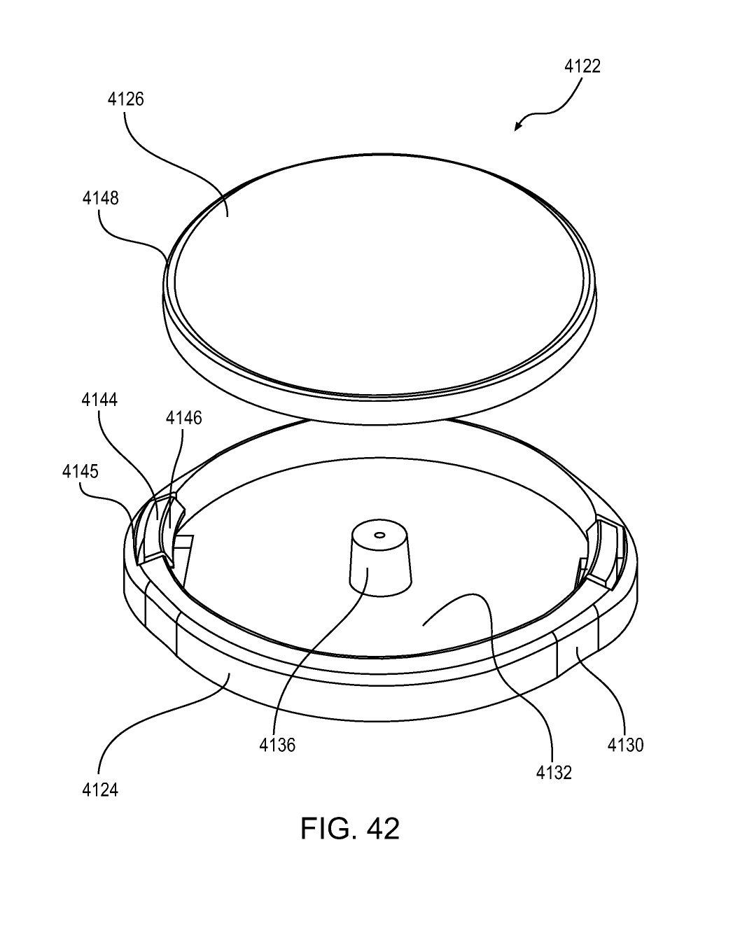

As illustrated in FIGS. 42-43, on body housing 4122 may be include two components, e.g., a mount component 4124 and an electronics component 4126. The underside 4128 of the mount component 4124 is adapted for placement on the skin of a patient, e.g., by use of an adhesive. It is also contemplated that mount component 4124 may be mounted to the skin with sutures or other techniques. The electronics component 4126 includes at least a portion of the sensor electronics 13/32 and its associated electronics 80, housed therein.

The mount component 4124 defines a surrounding wall 4130 and an upper surface 4132 in which the electronics component 4126 can be positioned. The electronics component 4126 defines a central aperture 4134 (not shown in FIG. 42 but substantially as illustrated in FIGS. 16-18 herein) which is configured for mounting over a central hub 4136. The central hub 4136 may be fabricated from a separate component and ultrasonically welded to the mount component 4124, or the central hub 4136 and mount component 4124 may be manufactured as a single element. The electronics component 4126 is provided with one or more contacts 4138 for providing electrical contact with contacts 4140 provided on the central hub 4136. A seal, such as an O-ring seal 4142, may be provided on the upper and lower portions of the electronics component 4126. Alternatively, the seals may be provided on the central hub 4136.

The electronics component 4126 is secured to the mount component in some embodiments by a snap-fit or friction-fit arrangement. In some embodiments, the mount component 4124 is provided with one or more snaps 4144 (two are shown in FIGS. 42 and 43), each having a engagement member 4146 which engages a circumferential groove 4148 provided on the top portion of the electronics component 4126. The snap 4144 is separated from the wall 4130 by a groove 4145. It is understood that the groove 4148 may be provided on the side walls of the electronics component 4126. In some embodiments, the groove 4148 may be replaced by indentations which correspond to the engagement members 4146 of the snaps 4144.

In some embodiments, the sensor 30 is partially disposed within the central hub 4136. As shown in FIG. 43, the insertion or internal portion 30 extends downwardly for insertion into the skin of the patient in contact with the ISF. The electrical contact portion 32 of the sensor 14 (not shown), which includes several conductive contacts, are connected to the contacts 4140 for connection to other electronics, e.g., at the sensor electronics 13/32 housed within the electronics component 4126. In the configuration shown in FIG. 43, the sensor may not have the bent configuration of FIG. 3, but rather have an upright configuration. As illustrated in FIGS. 16-18, an aperture provided in at least one of the electronics component 4126 and mount component 4124 allows the sharp 124 to extend therethrough.

As shown in FIG. 43, the electronics component 4126 is depressed downward (as indicated by the arrows) such that the snaps 4148 engage with the groove 4148. In some embodiments, the electronics component 4126 is attached to the mount component 4124 prior to insertion into the inserter assembly. For example, after attaching electronics component 4126 and mount component 4124, the combined on body housing 4122 may be inserted into the inserter assembly substantially as shown in FIGS. 22-26 in the same manner as on body housing 122. Similarly, the combined on body housing 4122 may be inserted into the inserter assembly substantially as shown in FIGS. 36-41 in the same manner as on body housing 122.

In some embodiments, the electronics component 4126 is inserted into the inserter assembly substantially as shown in FIGS. 22-26 and 36-41 in the same manner as on body housing 122. The mount component 4124 is attached to the skin of the patient in the desired location. Subsequently, the inserter assembly 100 or 2700 is placed over the mount component, and used to insert the sensor 30 into the skin of the patient, and advance the electronics component 4126 into engagement with the mount component 4124, as shown in FIG. 43.

In some embodiments, the electronics component 4126 is inserted into the inserter assembly substantially as shown in FIGS. 22-26 and 36-41 in the same manner as on body housing 122. The mount component 4124 is then advanced into engagement with the mount component 4124, as shown in FIG. 43, prior to attachment to the skin of the patient.

Another embodiment of the on body housing, referred to as on body housing is illustrated in FIG. 44. On body housing 4222 may be provided with a substantially circular configuration, or other shape, such as an elliptical or “football” shape. As with on body housing 122, on body housing 4222 has a reduced height (i.e., “Z”-dimension) to provide a low profile when sitting on the skin of the subject. In some embodiments, the height is about 3 mm to about 25 mm, e.g., may be about 4 mm, about 5 mm, about 10 mm, or about 15 mm. In certain embodiments, the on body housing 4222 may have a variable height such that it may have at least one portion that has a height that differs from at least one other portion. Electronics 80, for example, may include one or more of, e.g., an analog interface for connecting to the sensor 30, a processor, which may include, e.g., an ASIC, antenna, power supply to power the sensor and/or communications components, and capacitors.

As illustrated in FIG. 44, on body housing 4222 may include two components, e.g., a mount component 4224 and an electronics component 4226. The underside of the mount component 4224 (not shown in FIG. 44) is adapted for placement on the skin of a patient, e.g., by use of an adhesive. It is also contemplated that mount component 4224 may be mounted to the skin with sutures or other techniques. The electronics component 4226 includes at least a portion of the on body unit and its associated electronics 80, housed therein.

In some embodiments, the mount component 4224 defines a surrounding wall 4230, having one or more breaks or recesses 4250, and an upper surface 4232 in which the electronics component 4226 can be positioned. The electronics component 4226 defines a central aperture (not shown in FIG. 44, but substantially as illustrated in FIGS. 16-18 herein) which is configured for mounting over a central hub 4236. The central hub 4236 may be fabricated from a separate component and ultrasonically welded to the mount component 4224, or the central hub 4236 and mount component 4224 may be manufactured as a single element. The electronics component 4226 is provided with one or more contacts (not shown, see FIG. 43) for providing electrical contact with contacts (not shown, see FIG. 43) provided on the central hub 4236. A seal, such as an O-ring seal (not shown, see FIG. 43), may be provided on the upper and lower portions of the electronics component 4226. Alternatively, the seals may be provided on the central hub 4236.

The electronics component 4226 is secured to the mount component 4224 in some embodiments by a snap-fit or friction-fit arrangement. In some embodiments, the mount component 4224 is provided with one or more snaps 4244 (two are shown in FIG. 44), each having a engagement member 4246 which engages a circumferential groove 4248 provided on the top portion of the electronics component 4226. It is understood that the groove 4248 may be provided on the side walls of the electronics component 4226. In some embodiments, the groove 4248 may be replaced by indentations which correspond to the locations of the engagement members 4246 of the snaps 4244.

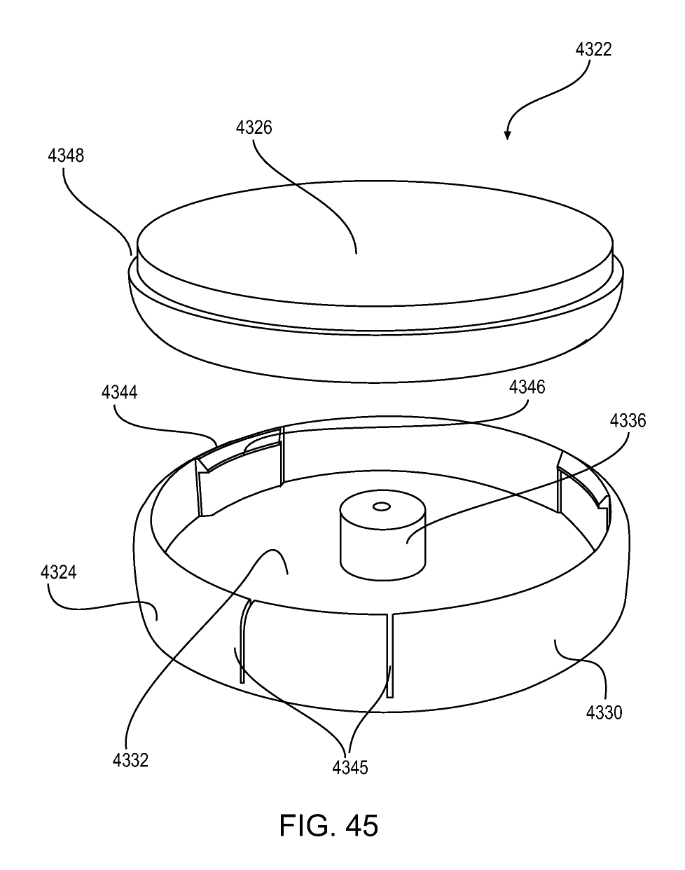

Another embodiment of the on body housing, referred to as on body housing is illustrated in FIG. 45. On body housing 4322 may be provided with a substantially circular configuration, or other shape, such as an elliptical or “football” shape. As with on body housing 122, on body housing 4322 has a reduced height (i.e., “Z”-dimension) to provide a low profile when sitting on the skin of the subject. In some embodiments, the height is about 3 mm to about 25 mm, e.g., may be about 4 mm, about 5 mm, about 10 mm, or about 15 mm. In certain embodiments, the on body housing 4322 may have a variable height such that it may have at least one portion that has a height that differs from at least one other portion. Electronics 80, for example, may include one or more of, e.g., an analog interface for connecting to the sensor 30, a processor, which may include, e.g., an ASIC, antenna, power supply to power the sensor and/or communications components, and capacitors.

As illustrated in FIG. 45, on body housing 4322 may include two components, e.g., a mount component 4324 and an electronics component 4326. The underside of the mount component 4324 (not shown in FIG. 45) is adapted for placement on the skin of a patient, e.g., by use of an adhesive. It is also contemplated that mount component 4324 may be mounted to the skin with sutures or other techniques. The electronics component 4326 includes at least a portion of the on body unit and its associated electronics 80, housed therein.

In some embodiments, the mount component 4324 defines a surrounding wall 4330 and an upper surface 4332 in which the electronics component 4326 can be positioned. The electronics component 4326 defines a central aperture (not shown in FIG. 45, but substantially as illustrated in FIGS. 16-18 herein) which is configured for mounting over a central hub 4336. The central hub 4336 may be fabricated from a separate component and ultrasonically welded to the mount component 4324, or the central hub 4336 and mount component 4324 may be manufactured as a single element. The electronics component 4326 is provided with one or more contacts (not shown, see FIG. 43) for providing electrical contact with contacts (not shown, see FIG. 43) provided on the central hub 4336. A seal, such as an O-ring seal (not shown, see FIG. 43), may be provided on the upper and lower portions of the electronics component 4326. Alternatively, the seals may be provided on the central hub 4336.

The electronics component 4326 is secured to the mount component 4324 in some embodiments by a snap-fit or friction-fit arrangement. In some embodiments, the mount component 4324 is provided with one or more snaps 4344 (three are shown in FIG. 45), each having a engagement member 4346 which engages a circumferential groove 4348 provided on the top portion of the electronics component 4326. A pair of grooves 4345 is provided in the wall 4330 in order to allow the snaps 4344 to flex radially inwardly and outwardly. It is understood that the groove 4348 may be provided on the side walls of the electronics component 4326. In some embodiments, the groove 4348 may be replaced by indentations which correspond to the locations of the engagement members 4346 of the snaps 4344.

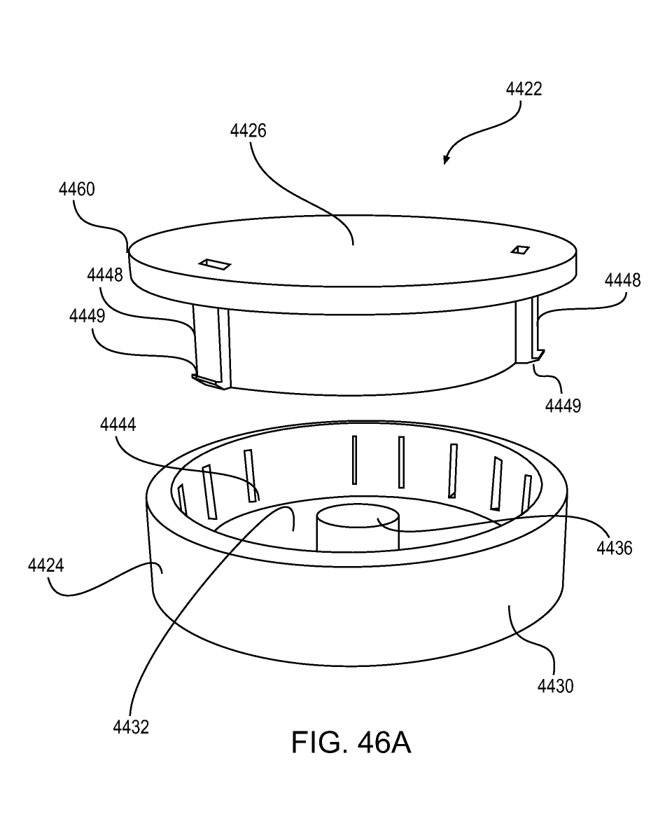

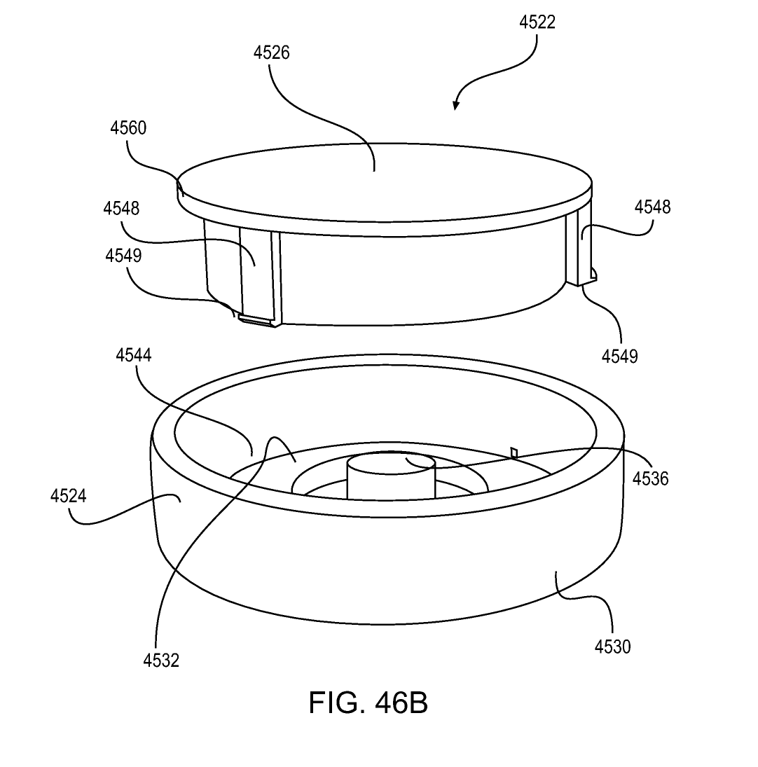

Another embodiment of the on body housing, referred to as on body housing is illustrated in FIG. 46A. On body housing 4422 may be provided with a substantially circular configuration, or other shape, such as an elliptical or “football” shape. As with on body housing 122, on body housing 4422 has a reduced height (i.e., “Z”-dimension) to provide a low profile when sitting on the skin of the subject. In some embodiments, the height is about 3 mm to about 25 mm, e.g., may be about 4 mm, about 5 mm, about 10 mm, or about 15 mm. In certain embodiments, the on body housing 4422 may have a variable height such that it may have at least one portion that has a height that differs from at least one other portion. Electronics 80, for example, may include one or more of, e.g., an analog interface for connecting to the sensor 30, a processor, which may include, e.g., an ASIC, antenna, power supply to power the sensor and/or communications components, and capacitors.

As illustrated in FIG. 46A on body housing 4422 may include two components, e.g., a mount component 4424 and an electronics component 4426. The underside of the mount component 4424 (not shown in FIG. 46A) is adapted for placement on the skin of a patient, e.g., by use of an adhesive. It is also contemplated that mount component 4424 may be mounted to the skin with sutures or other techniques. The electronics component 4426 includes at least a portion of the on body unit and its associated electronics 80, housed therein.

In some embodiments, the mount component 4424 defines a surrounding wall 4430 and an upper surface 4432 in which the electronics component 4426 can be positioned. The electronics component 4426 defines a central aperture (not shown in FIG. 46A, but substantially as illustrated in FIGS. 16-18 herein) which is configured for mounting over a central hub 4436. The central hub 4436 may be fabricated from a separate component and ultrasonically welded to the mount component 4424, or the central hub 4436 and mount component 4424 may be manufactured as a single element. The electronics component 4426 is provided with one or more contacts (not shown, see FIG. 43) for providing electrical contact with contacts (not shown, see FIG. 43) provided on the central hub 4436. A seal, such as an O-ring seal (not shown, see FIG. 43), may be provided on the upper and lower portions of the electronics component 4426. Alternatively, the seals may be provided on the central hub 4436.

The electronics component 4426 is secured to the mount component 4324 in some embodiments by a snap-fit or friction-fit arrangement. The electronics component 4426 may be provided with a circumferential rim 4460 which assists in the placement of the height of the electronics component 4426 relative to the mount component 4424. In some embodiments, the electronics component 4426 is provided with one or more snaps 4448 (two are shown in FIG. 46A), each having a engagement member 4449 which engages a circumferential groove 4444 provided along the inner surface of the wall 4430 of the mount component 4424. Snaps 4448 are flexible to flex radially inwardly and outwardly. In some embodiments, the groove 4444 may be replaced by indentations which correspond to the locations of the engagement members 4449 of the snaps 4448.

Another embodiment of the on body housing, referred to as on body housing is illustrated in FIG. 46B. On body housing 4522 may be provided with a substantially circular configuration, or other shape, such as an elliptical or “football” shape. As with on body housing 122, on body housing 4522 has a reduced height (i.e., “Z”-dimension) to provide a low profile when sitting on the skin of the subject. In some embodiments, the height is about 3 mm to about 25 mm, e.g., may be about 4 mm, about 5 mm, about 10 mm, or about 15 mm. In certain embodiments, the on body housing 4522 may have a variable height such that it may have at least one portion that has a height that differs from at least one other portion. Electronics 80, for example, may include one or more of, e.g., an analog interface for connecting to the sensor 30, a processor, which may include, e.g., an ASIC, antenna, power supply to power the sensor and/or communications components, and capacitors.

As illustrated in FIG. 46B on body housing 4522 may include two components, e.g., a mount component 4524 and an electronics component 4526. The underside of the mount component 4524 (not shown in FIG. 46A) is adapted for placement on the skin of a patient, e.g., by use of an adhesive. It is also contemplated that mount component 4524 may be mounted to the skin with sutures or other techniques. The electronics component 4526 includes at least a portion of the on body unit and its associated electronics 80, housed therein.

In some embodiments, the mount component 4524 defines a surrounding wall 4530 and an upper surface 4532 in which the electronics component 4526 can be positioned. The electronics component 4526 defines a central aperture (not shown in FIG. 46B, but substantially as illustrated in FIG. 43 herein) which is configured for mounting over a central hub 4536. The central hub 4536 may be fabricated from a separate component and ultrasonically welded to the mount component 4524, or the central hub 4536 and mount component 4524 may be manufactured as a single element. The electronics component 4526 is provided with one or more contacts (not shown, see FIG. 43) for providing electrical contact with contacts (not shown, see FIG. 43) provided on the central hub 4536. A seal, such as an O-ring seal (not shown, see FIG. 43), may be provided on the upper and lower portions of the electronics component 4526. Alternatively, the seals may be provided on the central hub 4536.

The electronics component 4526 is secured to the mount component 4524 in some embodiments by a snap-fit or friction-fit arrangement. The electronics component 4526 may be provided with a circumferential rim 4560 which assists in the placement of the height of the electronics component 4526 relative to the mount component 4524. In some embodiments, the electronics component 4526 is provided with one or more snaps 4548 (two are shown in FIG. 46B), each having a engagement member 4549 which engages a circumferential groove 4544 provided along the inner surface of the wall 4530 of the mount component 4524. Snaps 4548 are flexible to flex radially inwardly and outwardly. In some embodiments, the groove 4544 may be replaced by indentations which correspond to the locations of the engagement members 4549 of the snaps 4548.

### Inserter Assemblies

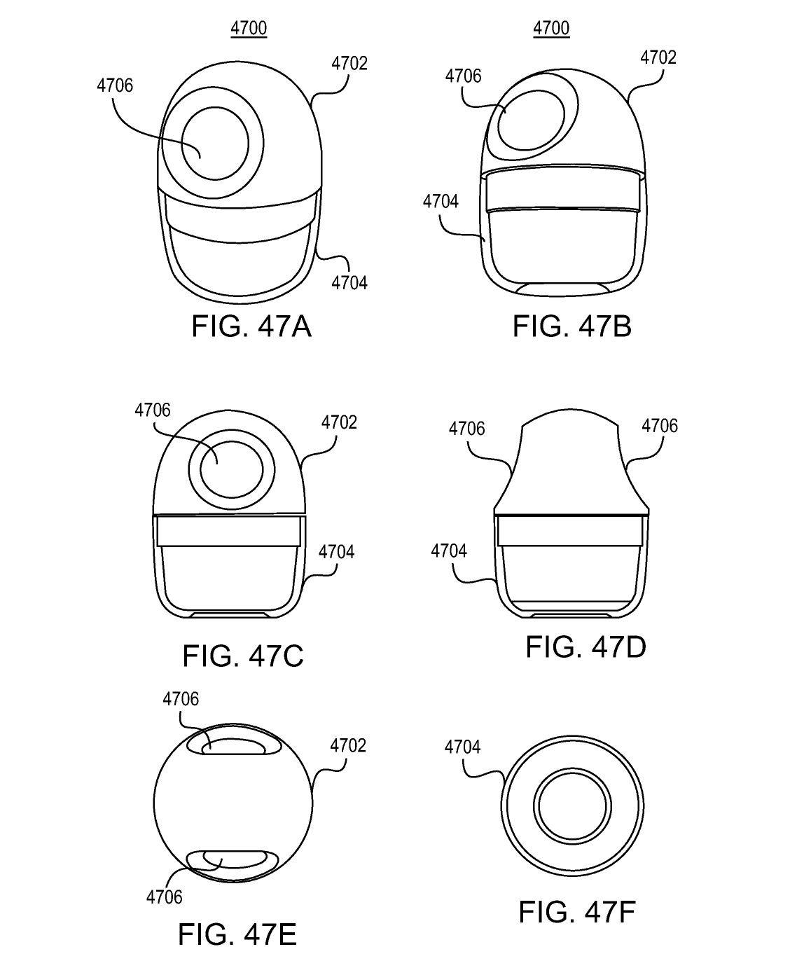

Additional embodiments of inserter assemblies similar to the inserter assembly discussed in conjunction with FIGS. 4-26 and FIGS. 27-41 are disclosed herein. Inserter assembly 4700, illustrated in FIGS. 47A-47F is similar to inserter assembly 2700 illustrated in FIGS. 27-41 with various aspects noted herein. For example, inserter assembly 4700 includes a housing 4702 and a cap 4704. In some embodiments, the housing 4702 and cap 4704 are separated by unscrewing the cap 4704 from the housing 4702, or by overcoming a friction fit. In the embodiment shown in FIGS. 47A-47F, the housing 4702 is provided with a substantially hemispherical configuration including two recesses 4706 for allowing the user to grip the housing 4702 for removal from the cap 4704 (lower portion).

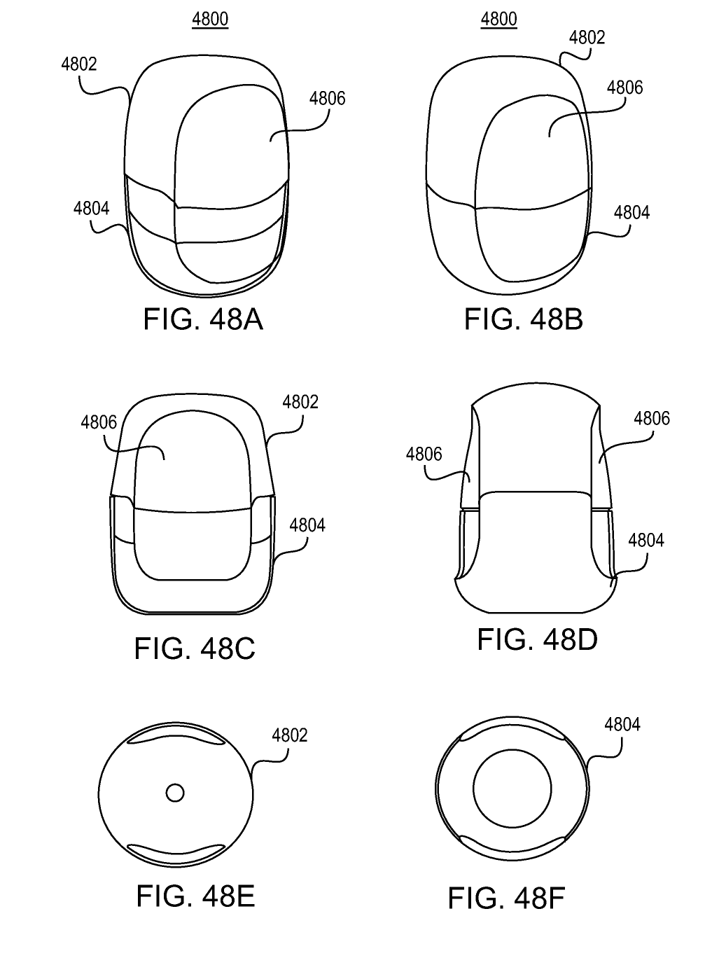

Inserter assembly 4800, illustrated in FIGS. 48A-48F is similar to inserter assembly 2700 illustrated in FIGS. 27-41 with various aspects noted herein. For example, inserter assembly 4800 includes a housing 4802 and cap 4804 having two recesses 4806 or flattened portions which extend from the upper housing portion 4802 to the lower cap portion 4804 for allowing the user to grip the housing 4802 for removal from the cap 4804 (lower portion).

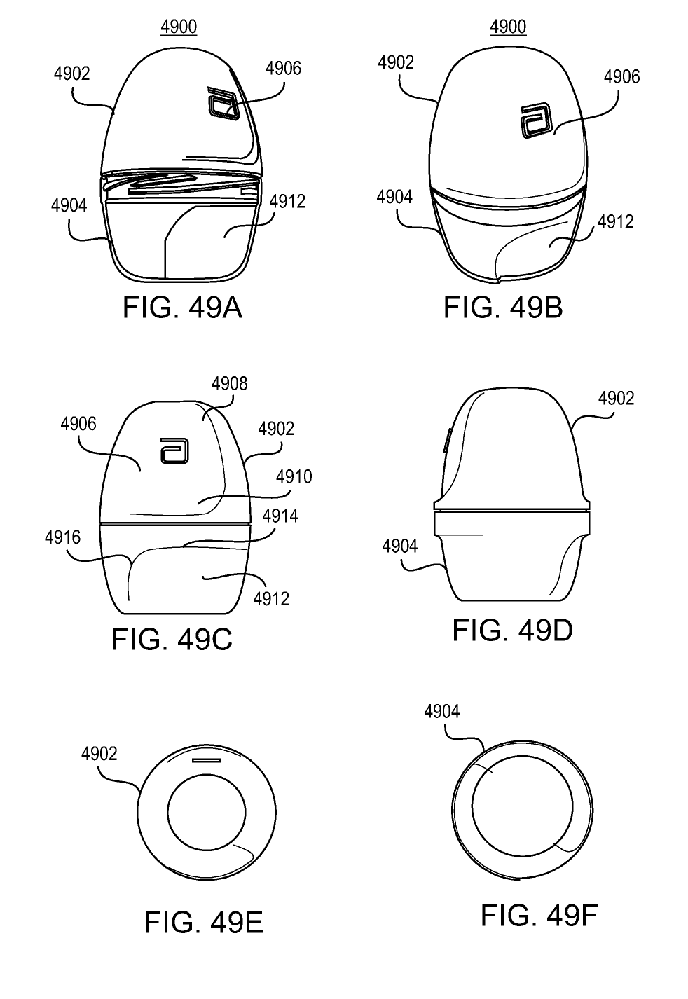

Inserter assembly 4900, illustrated in FIGS. 49A-49F is similar to inserter assembly 2700 illustrated in FIGS. 27-41 with various aspects noted herein. For example, inserter assembly 4900 includes a housing 4902 having a recessed portion 4906 defined by an L-shaped ridge (longitudinal portion 4908 and lateral portion 4910) and the cap portion 4904 includes a recessed portion 4912 defined by an L-shaped ridge (longitudinal portion 4916 and lateral portion 4914). The ridges are diametrically opposed to assist removal of the cap portion 4904 from the housing portion 4902.

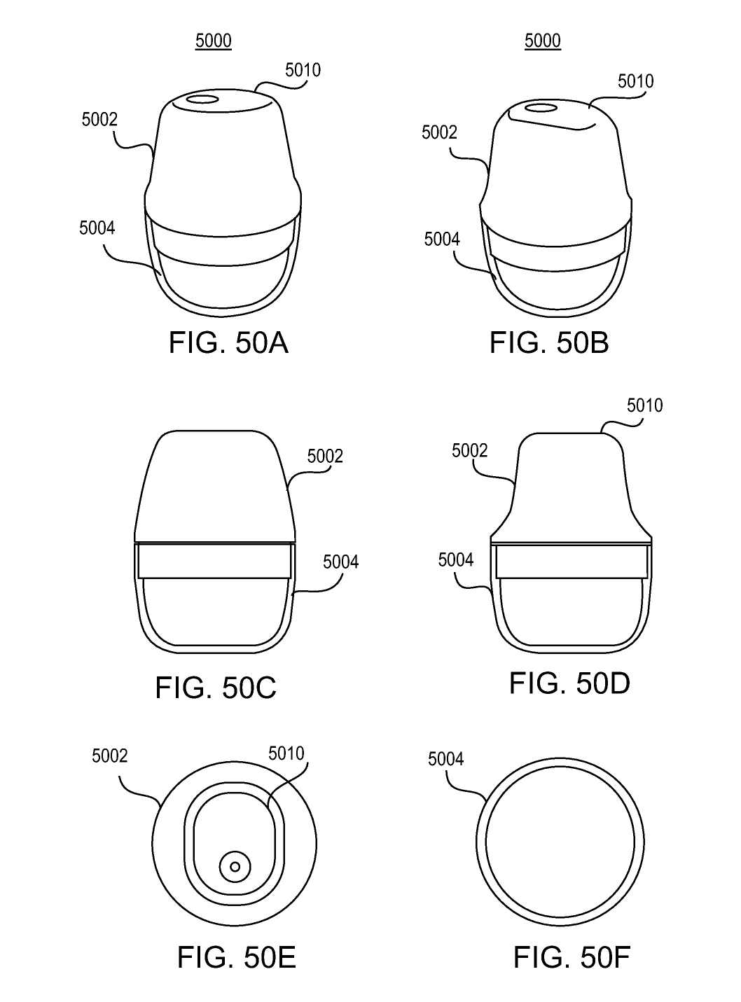

Inserter assembly 5000, illustrated in FIGS. 50A-50F is similar to inserter assembly 2700 illustrated in FIGS. 27-41 with various aspects noted herein. For example, inserter assembly 5000 includes a housing 5002 having a substantially oval or rectangular cross section at the proximal end portion 5010 (see FIG. 50E). This configuration assists removal of the cap portion 5004 from the housing portion 5002.

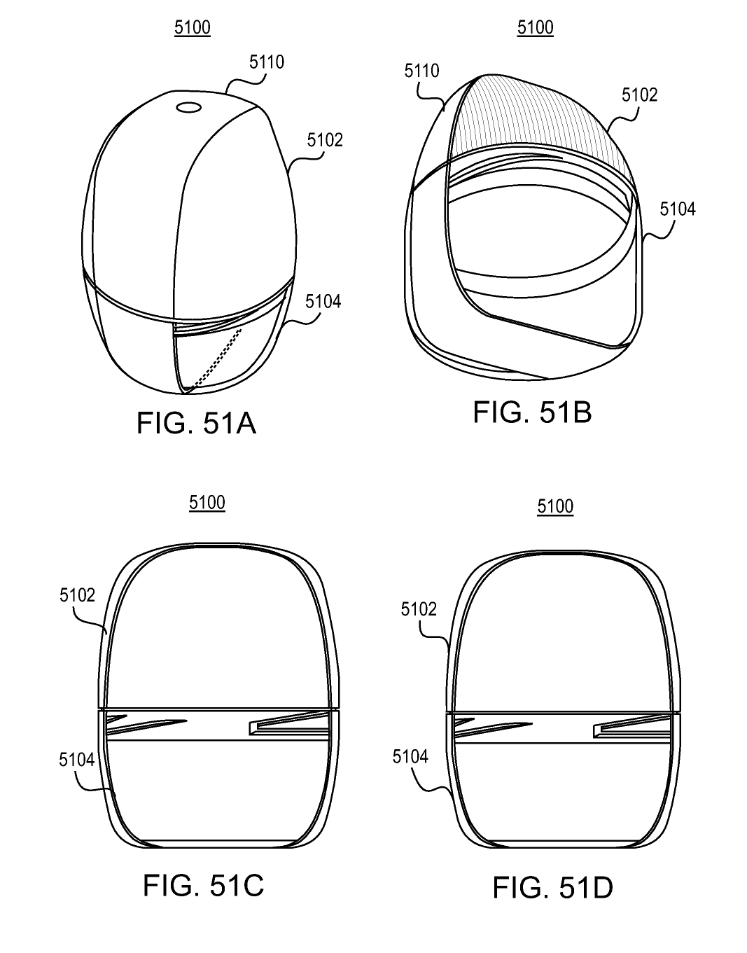

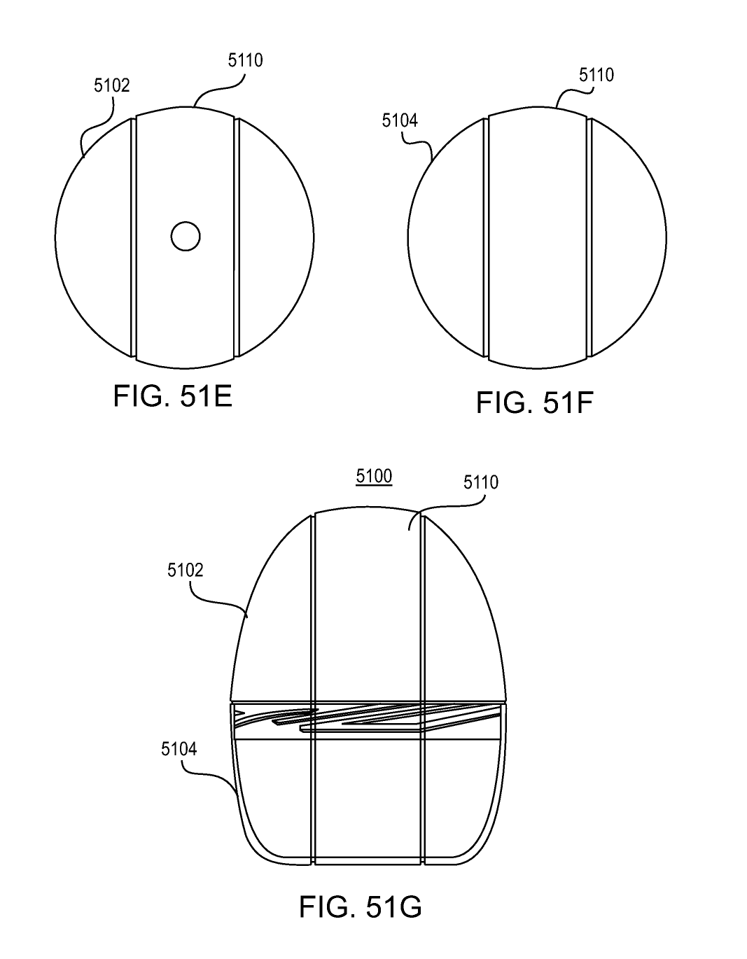

Inserter assembly 5100, illustrated in FIGS. 51A-51G is similar to inserter assembly 2700 illustrated in FIGS. 27-41 with various aspects noted herein. For example, inserter assembly 5100 includes a housing 5102 and a cap portion 5104 including a defined flattened band portion 5110. This configuration assists removal of the cap portion from the housing portion.

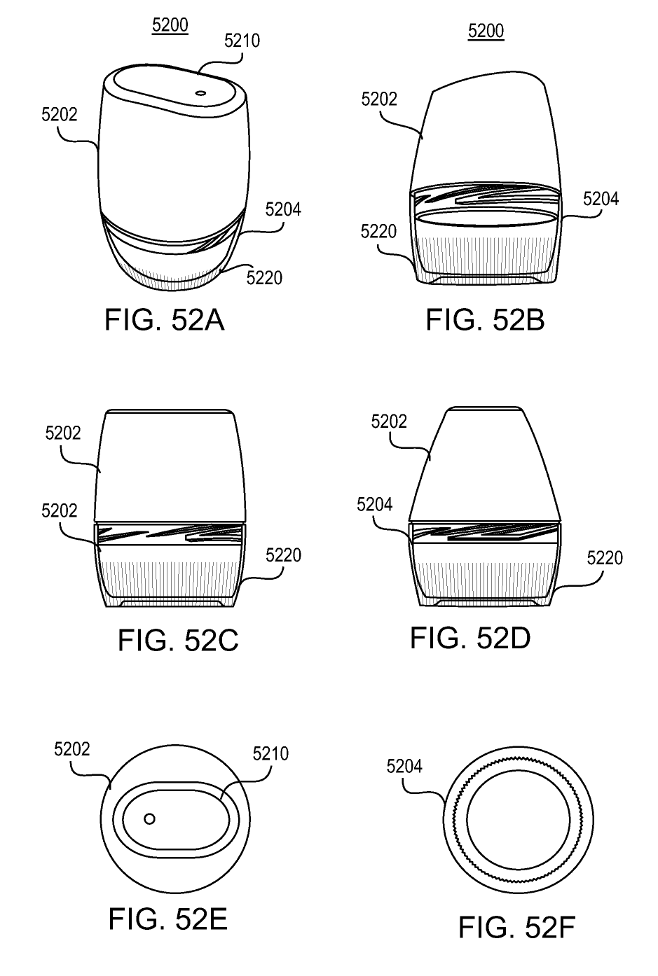

Inserter assembly 5200, illustrated in FIGS. 52A-52F is similar to inserter assembly 2700 illustrated in FIGS. 27-41 with various aspects noted herein. For example, inserter assembly 5200 includes a housing 5202 having a substantially oval or rectangular cross section at the proximal end portion 5210 (see FIG. 52E). This configuration assists removal of the cap portion 5204 from the housing portion 5202. Ridges 5220 are also provided on cap portion 5204 to facilitate removal of the cap portion 5204.

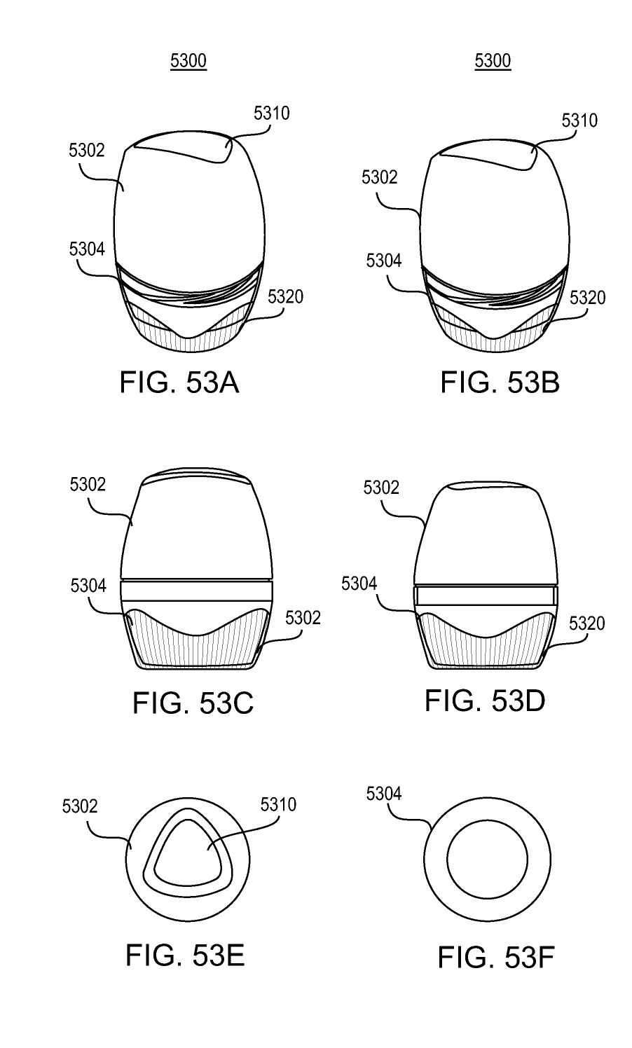

Inserter assembly 5300, illustrated in FIGS. 53A-53F is similar to inserter assembly 2700 illustrated in FIGS. 27-41 with various aspects noted herein. For example, inserter assembly 5300 includes a housing 5302 having a substantially triangular cross section at the proximal end portion 5310 (see FIG. 53E). This configuration assists removal of the cap portion 5304 from the housing portion 5302. Ridges 5320 are also provided on cap portion 5304 to facilitate removal of the cap portion 5304.

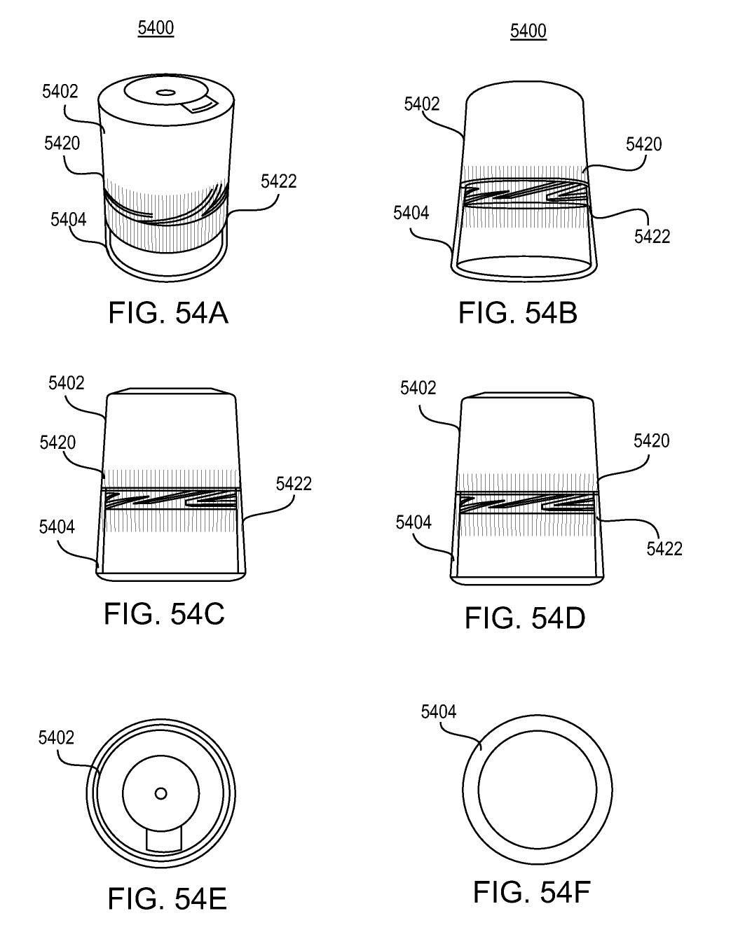

Inserter assembly 5400, illustrated in FIGS. 54A-54F is similar to inserter assembly 2700 illustrated in FIGS. 27-41 with various aspects noted herein. For example, inserter assembly 5400 includes a housing 5402 having ridges 5420 and a cap portion 5404 having ridges 5422 to facilitate removal of the cap portion 5404 from the housing portion 5402.

Embodiments of the present disclosure include a system for inserting an analyte sensor, comprising sensor electronics including a housing adapted for placement on a skin surface, an analyte sensor for monitoring an analyte level, and an electronics component provided in the housing and adapted to electrically couple to the sensor in the housing, and an inserter comprising a sheath defining a distal surface for placement on the skin surface, a device support movable between a proximal and distal position, and adapted to support the sensor electronics, a sharp support movable between a proximal and a distal position and adapted to support a sharp extending through a portion of said device support, a handle movable between a proximal position and a distal position relative to the sheath and adapted to urge the device support and the sharp support from a proximal to a distal position to insert the sharp and the analyte sensor into the skin, a driver for advancing the sharp support towards the proximal position when the sharp support reaches the distal position, wherein the driver includes at least one recess on an exterior surface thereof and a cap for coupling to the driver.

In certain aspects, the housing is provided with a circumferential wall for receiving the electronics component therein.

In certain aspects, the housing includes a central hub adapted to receive a portion of the sensor therein.

In certain aspects, the central hub includes electrical contacts for providing an electrical coupling between the sensor and the electronics component.

In certain aspects, the device support is adapted to support the housing of the sensor electronics.

In certain aspects, the driver and the cap are attached by one of a frictional fit, a snap fit, and complementary threads.

In certain aspects, at least one of the driver and the cap are provided with a frictional surface for separating the driver and the cap.

In certain aspects, the frictional surface comprises a flattened portion.

In certain aspects, the frictional surface comprises a ridge.

In certain aspects, the frictional surface comprises a plurality of ridges.

In certain aspects, the driver and the cap include at least one recess that extends from an upper portion of the driver to a lower portion of the cap when the driver and the cap are attached to each other.

In certain aspects, the cap includes at least one ridge.

In certain aspects, the at least one ridge of the driver and the cap is an L-shaped ridge.

In certain aspects, the L-shaped ridges of the driver and of the cap are diametrically opposed in orientation.

In certain aspects, the driver includes at least one of an oval, a rectangular, or a triangular cross-section.

In certain aspects, the cap includes a plurality of ridges formed in a longitudinal direction along a lower surface of the cap.

In certain aspects, the driver and the cap include at least one flattened portion that extends from an upper portion of the driver to a lower portion when the driver and the cap are attached to each other.

In certain aspects, the cap maintains a sterile environment for the sharp when coupled to the driver.

It is understood that the subject matter described herein is not limited to particular embodiments described, as such may, of course, vary. It is also understood that the terminology used herein is for the purpose of describing particular embodiments only, and is not intended to be limiting, since the scope of the present subject matter is limited only by the appended claims.

Additional detailed description of embodiments of the disclosed subject matter is provided in but not limited to: U.S. Pat. No. 7,381,184; U.S. Pat. No. 7,299,082; U.S. Pat. No. 7,167,818; U.S. Pat. No. 7,041,468; U.S. Pat. No. 6,942,518; U.S. Pat. No. 6,893,545; U.S. Pat. No. 6,881,551; U.S. Pat. No. 6,773,671; U.S. Pat. No. 6,764,581; U.S. Pat. No. 6,749,740; U.S. Pat. No. 6,746,582; U.S. Pat. No. 6,736,957; U.S. Pat. No. 6,730,200; U.S. Pat. No. 6,676,816; U.S. Pat. No. 6,618,934; U.S. Pat. No. 6,616,819; U.S. Pat. No. 6,600,997; U.S. Pat. No. 6,592,745; U.S. Pat. No. 6,591,125; U.S. Pat. No. 6,560,471; U.S. Pat. No. 6,540,891; U.S. Pat. No. 6,514,718; U.S. Pat. No. 6,514,460; U.S. Pat. No. 6,503,381; U.S. Pat. No. 6,461,496; U.S. Pat. No. 6,377,894; U.S. Pat. No. 6,338,790; U.S. Pat. No. 6,299,757; U.S. Pat. No. 6,284,478; U.S. Pat. No. 6,270,455; U.S. Pat. No. 6,175,752; U.S. Pat. No. 6,161,095; U.S. Pat. No. 6,144,837; U.S. Pat. No. 6,143,164; U.S. Pat. No. 6,134,461;U.S. Pat. No. 6,121,009; U.S. Pat. No. 6,120,676; U.S. Pat. No. 6,071,391; U.S. Pat. No. 5,918,603; U.S. Pat. No. 5,899,855; U.S. Pat. No. 5,822,715; U.S. Pat. No. 5,820,551; U.S. Pat. No. 5,628,890; U.S. Pat. No. 5,601,435; U.S. Pat. No. 5,593,852; U.S. Pat. No. 5,509,410; U.S. Pat. No. 5,320,715; U.S. Pat. No. 5,264,014; U.S. Pat. No. 5,262,305; U.S. Pat. No. 5,262,035; U.S. Pat. No. 4,711,245; U.S. Pat. No. 4,545,382; U.S. Patent Publication No. 2004/0186365, published Sep. 23, 2004; U.S. Patent Application No. 61/238,646, filed Aug. 31, 2009; U.S. patent application Ser. No. 12/698,129, filed Feb. 1, 2010; U.S. Patent Application No. 61/317,243, filed Mar. 24, 2010; U.S. Patent Application No. 61/345,562, filed May 17, 2010; International Application No. PCT/US10/22860, filed Feb. 22, 2010; and U.S. patent application Ser. No. 12/807,278, filed Aug. 31, 2010, the disclosures of each of which is incorporated by reference herein for all purposes.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

What is claimed is:

1. A system for inserting an analyte sensor, comprising:
sensor electronics including a housing adapted for placement on a skin surface, an analyte sensor for monitoring an analyte level, and an electronics component provided in the housing and adapted to electrically couple to the sensor in the housing; and
an inserter comprising:
a sheath defining a distal surface for placement on the skin surface;
a device support movable between a proximal and distal position, and adapted to support the sensor electronics;
a sharp support movable between a proximal and a distal position and adapted to support a sharp extending through a portion of said device support;
a handle movable between a proximal position and a distal position relative to the sheath and adapted to urge the device support and the sharp support from a proximal to a distal position to insert the sharp and the analyte sensor into the skin;
a driver for advancing the sharp support towards the proximal position when the sharp support reaches the distal position, wherein the driver includes at least one recess on an exterior surface thereof; and
a cap for coupling to the driver.

2. The system of claim 1, wherein the housing is provided with a circumferential wall for receiving the electronics component therein.

3. The system of claim 1, wherein the housing includes a central hub adapted to receive a portion of the sensor therein.

4. The system of claim 3, wherein the central hub includes electrical contacts for providing an electrical coupling between the sensor and the electronics component.

5. The system of claim 1, wherein the device support is adapted to support the housing of the sensor electronics.

6. The system of claim 1, wherein the driver and the cap are attached by one of a frictional fit, a snap fit, and complementary threads.

7. The system of claim 6, wherein at least one of the driver and the cap are provided with a frictional surface for separating the driver and the cap.

8. The system of claim 7, wherein the frictional surface comprises a flattened portion.

9. The system of claim 7, wherein the frictional surface comprises a ridge.

10. The system of claim 7, wherein the frictional surface comprises a plurality of ridges.

11. The system of claim 1, wherein the driver and the cap include at least one recess that extends from an upper portion of the driver to a lower portion of the cap when the driver and the cap are attached to each other.

12. The system of claim 1, wherein the cap includes at least one ridge.

13. The system of claim 12, wherein the at least one ridge of the driver and the cap is an L-shaped ridge.

14. The system of claim 13, wherein the L-shaped ridges of the driver and of the cap are diametrically opposed in orientation.

15. The system of claim 1, wherein the driver includes at least one of an oval, a rectangular, or a triangular cross-section.

16. The system of claim 1, wherein the cap includes a plurality of ridges formed in a longitudinal direction along a lower surface of the cap.

17. The system of claim 1, wherein the driver and the cap include at least one flattened portion that extends from an upper portion of the driver to a lower portion when the driver and the cap are attached to each other.

18. The system of claim 1, wherein the cap maintains a sterile environment for the sharp when coupled to the driver.
